(19) United States
(12) Patent Application Publication
(10) Pub. No.: US 2011/0183344 A1
(43) Pub. Date: Jul. 28, 2011

(54) COMPOSITIONS FOR USE IN IDENTIFICATION OF CLOSTRIDIUM DIFFICILE

(76) Inventors: Rangarajan Sampath, San Diego, CA (US); Feng Li, San Diego, CA (US); David J. Ecker, Encinitas, CA (US); Robert J. Lovari, San Marcos, CA (US); Lawrence B. Blyn, Mission Viejo, CA (US); Thomas A. Hall, Oceanside, CA (US)

(21) Appl. No.: 13/122,364

(22) PCT Filed: Sep. 30, 2009
(86) PCT No.: PCT/US09/58960

§ 371 Date: Apr. 1, 2011

(51) Int. Cl. C12Q 1/68; C07H 21/00; H01J 49/26

(52) U.S. Cl. 435/6.12; 536/23.1; 250/281

(57) ABSTRACT

The present invention relates generally to identification of strains of Clostridium difficile and provides methods, compositions and kits useful for this purpose when combined, for example, with molecular mass or base composition analysis.

### CROSS REFERENCE TO RELATED APPLICATIONS

The present application claims priority to U.S. Provisional Application No. 61/102,679, filed Oct. 3, 2008, which is incorporated by reference in its entirety.

### FIELD OF THE INVENTION

The present invention relates generally to the identification of Clostridium difficile and particular strains thereof. The invention provides methods, compositions and kits useful for this purpose when combined, for example, with molecular mass or base composition analysis.

### BACKGROUND OF THE INVENTION

Clostridium difficile is a species of bacteria of the genus Clostridium which are Gram-positive, anaerobic, spore-forming rods (bacillus). Clostridia are motile bacteria that are ubiquitous in nature and are especially prevalent in soil. Clostridium difficile cells show optimum growth on blood agar at human body temperatures in the absence of oxygen. When stressed, Clostridium difficile bacteria produce spores which enable this bacterium to tolerate extreme conditions.

C. difficile is a commensal bacterium of the human intestine in a minority of the population, especially in patients undergoing long-term care in a hospital or a nursing home. In small numbers, it does not result in disease of significance.

With the introduction of broad-spectrum antibiotics in the latter half of the twentieth century, antibiotic-associated diarrhea became more common. Antibiotics, especially those with a broad spectrum of activity, cause disruption of normal intestinal flora, leading to an overgrowth of C. difficile. This may lead to pseudomembranous colitis. Pseudomembranous colitis caused by C. difficile is treated with specific antibiotics, for example, vancomycin, metronidazole, bacitracin or fusidic acid.

C. difficile is resistant to many antibiotics. It is transmitted from person to person by the fecal-oral route. Because the organism forms heat-resistant spores, it is able to persist in the environment for long periods of time. Once spores are ingested, they often pass through the stomach unscathed because of their acid-resistance. They change to their active form in the colon and multiply.

The strain BI/NAP1 has contributed to outbreaks in several regions of North America, the United Kingdom, and the Netherlands. It was identified and referred to by polymerase chain reaction as ribotype 27 (CD027), by pulsed-field gel electrophoresis as North American pulse-field type 1 (NAP1), by restriction-endonuclease analysis (REA) as group BI, and by toxinotyping as toxinotype III. Various publications may refer to the strain by different terms including BI/NAP1 or NAP1/027 but they are referring to the same strain. This current outbreak strain is distinct from another strain which caused previous outbreaks during the late 1980s and early 1990s, known as the J strain (REA type J7/9). As previously mentioned, notable virulence characteristics associated with the BI/NAP1 strain include increased toxin production (tcdA and tcdB), the presence of a binary toxin, altered antimicrobial resistance patterns (fluoroquinolone resistance), and increased sporulation capacity.

The tcdA toxin has been termed “the enterotoxin,” as it is responsible for the expression of diarrhea and colonic inflammation. The tcdB toxin is known as “the cytotoxin” and is responsible for actinomorphic changes in tissue culture cells. In all but rare cases, both toxins are expressed in patients with clinical disease; however, strains that are tcdA negative but tcdB positive have been identified. The toxins TcdA (308 kD) and TcdB (270 kD) are among the largest toxins to be harbored by bacteria and are encoded on a chromosome within the pathogenicity locus (PaLoc) of the organism. Also located within the PaLoc are regulatory genes such as tcdC, which is a downstream negative regulatory gene that controls the expression of tcdA and tcdB. All identified BI/NAP1 strains contain an 18-base pair tcdC gene deletion that is thought to be responsible for the accelerated kinetics of toxin production. Traditionally, tcdA and tcdB are produced most efficiently when the organism is in the stationary growth phase. In contrast, BI/NAP1 strains produce 16 times more tcdA and 23 times more tcdB, and studies indicate that most of this production occurs in the logarithmic growth phase.

In addition to the well-characterized tcdA and tcdB toxins, a formerly uncommon binary toxin has been identified in all of the BI/NAP1 isolates (previously found in approximately 6% of clinical isolates). The structure and function of this toxin are similar to those of other binary toxins, such as iota toxin found in Clostridium perfringens. Although patients infected with binary toxin-positive strains of Clostridium difficile trended toward having greater disease severity, tcdA and tcdB-negative but binary toxin-positive strains of C. difficile have been shown to be nonpathogenic in classic nonclinical models of infection. Thus, the pathogenic role of binary toxin in BI/NAP1 is unknown.

Clostridium difficile strains can also be classified by toxinotyping studies. Subtle sequence variations within the PaLoc accounts for the various toxinotypes of C. difficile, of which there have been reported to be at least 22 different types. Toxinotype III, to which BI/NAP1 belongs, was previously rare, accounting for only 2-3% of clinical isolates. Whether toxinotyping can be used to distinguish virulence potential among strains has yet to be demonstrated.

Genotypically distinct strains of C. difficile have been shown to demonstrate a propensity to hypersporulate and have been reported to be responsible for previous outbreaks. The BI/NAP1 strain, like other outbreak strains, has demonstrated the capacity to hypersporulate compared with other nonoutbreak strains. This putative virulence characteristic may be, at least in part, responsible for its rapid establishment in many institutions where outbreaks have been reported. As with the other recently identified characteristics of this organism, future studies are required to elucidate the exact role of hypersporulation in the transmission or pathology of C. difficile.

Clostridium difficile infection (CDI) may range in severity from asymptomatic to severe and life threatening, and many deaths have been reported, especially amongst the aged. People are most often infected in hospitals, nursing homes, or institutions, although C. difficile infection in community and outpatient settings is increasing. Clostridium difficile associated diarrhea (aka CDAD) has been linked to use of broad-spectrum antibiotics such as cephalosporins and clindamycin, though the use of quinolones is now a likely culprit. The incidence and severity of C. difficile colitis remains high and is often seen in association with elevated death rates. Immunocompromised patient status and delayed diagnosis often result in enhanced risk of death. Early intervention and aggressive management are key factors to recovery.

Three antibiotics are effective against C. difficile. Metronidazole is often the drug of choice, because of lower price and acceptable efficacy. Oral vancomycin is a second-line therapy, but may be avoided due to concerns regarding conversion of intestinal flora into vancomycin-resistant organisms. However, it may be used, for example, in the following cases: severe C. difficile diarrhea; no response to oral metronidazole; the organism is resistant to metronidazole; the patient is allergic to metronidazole; and when the patient is either pregnant or younger than 10 years of age. For these purposes vancomycin is often administered orally because IV administration does not achieve gut lumen minimum therapeutic concentration. The use of linezolid may also be considered.

What is needed are improved methods of diagnosing and characterizing C. difficile infections, in particular, methods of distinguishing virulent from non-virulent strains.

### SUMMARY OF THE INVENTION

The present invention relates generally to the detection and identification of strains of Clostridium difficile and provides methods, compositions and kits useful for this purpose when combined, for example, with molecular mass or base composition analysis.

In some embodiments, the present invention relates to identification of strains of Clostridium difficile in, for example, a single sample from a patient, and provides methods, compositions and kits useful for this purpose. However, the compositions and methods described above find use in a variety of biological sample analysis techniques and are not limited to processes that employ or require molecular mass or base composition analysis. For example, primers described herein find use in a variety of research, surveillance, and diagnostic approaches that utilize one or more primers, including a variety of approaches that employ the polymerase chain reaction.

To further illustrate, in certain embodiments the invention provides for the rapid detection and characterization of strains of Clostridium difficile. The primer pairs described herein, for example, may be used to detect specific strains of Clostridium difficile. In addition to compositions and kits that include one or more of the primer pairs described herein, the invention also provides related methods and systems.

In one aspect, there is provided a purified oligonucleotide primer pair for identifying a strain of Clostridium difficile in a sample. The primer pair includes a forward primer and a reverse primer, each configured to hybridize to nucleic acid of two or more different strains of Clostridium difficile in a nucleic acid amplification reaction which produces an amplification product between about 29 to about 200 nucleobases in length. The amplification product includes portions corresponding to a forward primer hybridization region, a reverse primer hybridization region and an intervening region having a base composition which varies among amplification products produced from nucleic acid of the two or more different strains of Clostridium difficile. The base composition of the intervening region provides a means for identifying the strain of Clostridium difficile.

In certain embodiments of the primer pair, the strain of Clostridium difficile includes a subspecies characteristic selected from the group consisting of: binary toxin cdtA, and binary toxin cdtB, toxin A, toxin B, an 18-nucleobase 18 nucleobase deletion within the tcdC gene, and a single nucleobase deletion within the tcdC gene.

In certain embodiments of the primer pair, each member of the primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

In certain embodiments of the primer pair, the forward primer and the reverse primer are about 14 to about 40 nucleobases in length.

In certain embodiments of the primer pair, the forward primer or the reverse primer or both further include a non-templated thymidine residue on the 5′-end, a mass modifying tag, a modified nucleobase, preferably 5-propynyluracil or 5-propynylcytosine, a mass-modified nucleobase, preferably 5-iodo-cytosine, or a universal nucleobase, preferably inosine.

Another aspect of the invention is an isolated amplification product for identification of a strain of Clostridium difficile. The amplification product is produced by a process which includes the step of amplifying nucleic acid of the strain of Clostridium difficile in a reaction mixture which includes a primer pair. The primer pair includes a forward primer and a reverse primer, each configured to hybridize to nucleic acid of two or more different bacteria in a nucleic acid amplification reaction. The amplification product has a length of about 29 to about 200 nucleobases and includes portions corresponding to a forward primer hybridization region, a reverse primer hybridization region and an intervening region having a base composition which varies among amplification products produced from nucleic acid of the two or more different strains of Clostridium difficile. The base composition of the intervening region provides a means for identifying the strain of Clostridium difficile. Also included in the process is the step of isolating the amplification product from the reaction mixture. In certain embodiments of the amplification product, the isolating step is performed using an anion exchange resin linked to a magnetic bead.

In certain embodiments of the amplification product, each member of the primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

In certain embodiments of the amplification product, the forward primer and the reverse primer are about 14 to about 40 nucleobases in length.

In certain embodiments of the amplification product, the forward primer or the reverse primer or both further include a non-templated thymidine residue on the 5′-end, a mass modifying tag, a modified nucleobase, preferably 5-propynyluracil or 5-propynylcytosine, a mass-modified nucleobase, preferably 5-iodo-cytosine, or a universal nucleobase, preferably inosine.

Another aspect of the invention is a method for identifying a strain of Clostridium difficile. The method includes the step of obtaining an amplification product by amplifying nucleic acid of a bacterium in the sample using the primer pair embodiments described above. The molecular mass of one or both strands of the amplification product are measured and then compared to a plurality of database-stored molecular masses of strands of amplification products of known strains of Clostridium difficile. The identification of a match between the molecular mass and at least one of the database-stored molecular masses of amplification products identifies the strain of Clostridium difficile.

In another aspect of the invention, there is provided a method for identifying a strain of Clostridium difficile in a sample. The method includes the step of obtaining an amplification product by amplifying nucleic acid of a strain of Clostridium difficile in the sample using an embodiment of the purified primer pair described above. The molecular mass of one or both strands of the amplification product is measured. The base composition of the amplification product is determined from the molecular mass. The base composition is then compared to a plurality of database-stored base compositions of strands of amplification products of known bacteria. The identification of a match between the base composition and at least one of the database-stored base compositions of amplification products identifies the strain of Clostridium difficile.

In certain embodiments of the method, each member of the primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

In certain embodiments of the method, the molecular mass is determined by mass spectrometry.

Another aspect of the invention is a kit which includes one or more purified primer pairs for identifying a strain of Clostridium difficile in a sample. Each member of the one or more primer pairs has at least 70% sequence identity with a corresponding member of one or more primer pairs selected from the group consisting of: SEQ ID NOs: 18:19, 8:25, 21:2, 17:23, 14:10, 24:7, 22:5, 3:11, 13:9, 12:20, 15:6, 16:6 and 1:4.

The kit may also include deoxynucleotide triphosphates, preferably ¹³C-enriched deoxynucleotide triphosphates.

In another aspect of the invention, there is provided a system which includes a mass spectrometer configured to detect one or more molecular masses of embodiments of the amplification product described above. The system also includes a database of known molecular masses and/or known base compositions of amplification products of strains of Clostridium difficile. A controller is operably connected to the mass spectrometer and to the database. The controller is configured to match the molecular masses of the amplification product with a measured or calculated molecular mass of a corresponding amplification product of a strain of Clostridium difficile.

In certain embodiments of the system, the database of known molecular masses and/or known base compositions of amplification products includes amplification products defined by one or more primer pairs that have members with at least 70% sequence identity with a corresponding member of a corresponding primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

### BRIEF DESCRIPTION OF THE DRAWINGS

The foregoing summary and detailed description is better understood when read in conjunction with the accompanying drawings which are included by way of example and not by way of limitation.

FIG. 1 shows a process diagram illustrating one embodiment of the primer pair selection process.

FIG. 2 shows a process diagram illustrating one embodiment of the primer pair validation process. Criteria include but are not limited to, the ability to amplify nucleic acid of strains of Clostridium difficile, the ability to exclude amplification of extraneous nucleic acids and dimerization of primers, analytical limits of detection of 100 or fewer genomic copies/reaction, and the ability to strains of Clostridium difficile.

FIG. 3 shows a process diagram illustrating an embodiment of the calibration method.

FIG. 4 shows a block diagram showing a representative system.

### DETAILED DESCRIPTION OF EMBODIMENTS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains.

In describing and claiming the present invention, the following terminology and grammatical variants will be used in accordance with the definitions set forth below.

As used herein, the term “about” means encompassing plus or minus 10%. For example, the term “about 200 nucleotides” is with reference to a range encompassing between 180 and 220 nucleotides.

As used herein, the term “amplicon” or “bioagent identifying amplicon” refers to a nucleic acid segment deduced from hybridization of primer pairs to a known nucleic acid sequence. The deduction of an amplicon is well within the capabilities of a person skilled in the art. An amplicon may, for example, be deduced on a page containing the known nucleic acid sequence and the sequences of the primers or may be deduced using in silico methods such as electronic PCR which are known to the skilled person. The skilled person will also readily recognize that the amplicon contains primer hybridization portions and an intervening portion between the two primer hybridization portions. One important objective is to define many bioagent identifying amplicons using as few primer pairs as possible. Another important objective is to provide a primer pair which is specific for particular strains of Clostridium difficile.

As used herein, the term “amplicon” or “bioagent identifying amplicon” is distinct from the term “amplification product” in that the term “amplification product” refers to the physical biomolecule produced in an actual amplification reaction. With respect to these definitions, an amplification product “corresponds” to an amplicon. This means that an amplicon may be present in silico in a database even prior to a corresponding amplification product ever being produced in an amplification reaction. An amplification product which corresponds to an amplicon must be produced by the same primers used to deduce the amplicon. The skilled person will recognize that if an amplicon residing in a database is in the form of a DNA sequence, an RNA sequence may be readily deduced from it, or vice versa. Thus, in the case of an RNA sequence, a DNA sequence of an amplicon may be deduced from the RNA sequence for any given primer pair.

The amplification products are typically double stranded DNA; however, it may be RNA and/or DNA:RNA. In some embodiments, the amplification product comprises sequences of conserved regions/primer pairs and intervening variable region. As discussed herein, primer pairs are configured to generate amplification products from nucleic acid of strains of Clostridium difficile. As such, the base composition of any given amplification product includes the base composition of each primer of the primer pair, the complement of each primer the primer pair and the intervening variable region from the bioagent that was amplified to generate the amplification product. One skilled in the art understands that the incorporation of the designed primer pair sequences into an amplification product may replace the native sequences at the primer binding site, and complement thereof. In certain embodiments, after amplification of the target region using the primers the resultant amplification product having the primer sequences are used to generate the molecular mass data. Generally, the amplification product further comprises a length that is compatible with mass spectrometry analysis. The amplification products corresponding to bioagent identifying amplicons have base compositions that are preferably unique to the identity of a bioagent such as strains of Clostridium difficile.

Amplicons and amplification products typically comprise from about 29 to about 200 consecutive nucleobases (i.e., from about 29 to about 200 linked nucleosides). One of ordinary skill in the art will appreciate that this range expressly embodies compounds of 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One of ordinary skill in the art will further appreciate that the above range is not an absolute limit to the length of an amplicon and amplification product, but instead represents a preferred length range. Lengths of amplification products falling outside of this range are also included herein so long as the amplification product is amenable to experimental determination of its molecular mass and/or its base composition as herein described.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “base composition” refers to the number of each residue in an amplicon, amplification product or other nucleic acid, without consideration for the linear arrangement of these residues in the strand(s). The residues may comprise, adenosine (A), guanosine (G), cytidine, (C), (deoxy)thymidine (T), uracil (U), inosine (I), nitroindoles such as 5-nitroindole or 3-nitropyrrole, dP or dK (Hill F et al. Polymerase recognition of synthetic oligodeoxyribonucleotides incorporating degenerate pyrimidine and purine bases. Proc Natl Acad Sci USA. 1998 Apr. 14; 95(8):4258-63), an acyclic nucleoside analog containing 5-nitroindazole (Van Aerschot et al., Nucleosides and Nucleotides, 1995, 14, 1053-1056), the purine analog 1-(2-deoxy-beta-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. Herein the base composition is notated as A_{w}GₓC_{y}T_{z}, wherein w, x, y and z are each independently a whole number representing the number of the nucleoside residues in an amplicon and wherein T (thymidine) may be replaced by uracil (U) if desired, by simply using uridine triphosphates in the amplification reaction.

Base compositions of amplification products which include modified nucleosides are similarly notated to indicate the number of the natural and modified nucleosides in an amplification product. Base compositions are determined from a molecular mass measurement of an amplification product, as described below. The base composition for any given amplification product is then compared to a database of base compositions which typically includes base compositions calculated from sequences of amplicons deduced from a given primer pair and the known hybridization coordinates of the primers of the primer pair on the specific nucleic acid of strains of Clostridium difficile. A match between the base composition of the amplification product and a single database amplicon entry reveals the identity of the bioagent. Alternatively, if a match between the base composition of the amplification product and the base compositions of individual amplicons in the database is not obtained, the conclusion may be drawn that the amplification product was obtained from nucleic acid of a previously uncharacterized strain of Clostridium difficile which may contain one or more SNPs, deletions, insertions or other sequence variations within the intervening variable region between the two primer hybridization sites. This is useful information which characterizes the previously uncharacterized strain of Clostridium difficile. It is useful to then incorporate the base composition of the previously uncharacterized strain of Clostridium difficile into the base composition database.

As used herein, a “base composition probability cloud” is a representation of the diversity in base composition resulting from a variation in sequence that occurs among different isolates of a given species, family or genus. Base composition calculations for a plurality of amplicons are mapped on a pseudo four-dimensional plot. Related members in a family, genus or species typically cluster within this plot, forming a base composition probability cloud.

As used herein, the term “base composition signature” refers to the base composition generated by any one particular amplicon.

As used herein, a “bioagent” means any biological organism or component thereof or a sample containing a biological organism or component thereof, including microorganisms or infectious substances, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Still, a non-exhaustive list of bioagents includes: cells, cell lines, human clinical samples, mammalian blood samples, cell cultures, bacterial cells, viruses, viroids, fungi, protists, parasites, Rickettsiae, protozoa, animals, mammals or humans. Samples may be alive, non-replicating or dead or in a vegetative state (for example, vegetative bacteria or spores). Preferably, the bioagent is a strain of Clostridium difficile.

As used herein, a “bioagent division” is defined as group of bioagents above the species level and includes but is not limited to, orders, families, genus, classes, clades, genera or other such groupings of bioagents above the species level.

As used herein, “broad range survey primers” are primers designed to identify an unknown bioagent as a member of a particular biological division (e.g., an order, family, class, clade, or genus). However, in some cases the broad range survey primers are also able to identify unknown bioagents at the species or sub-species level. As used herein, “division-wide primers” are primers designed to identify a bioagent at the species level and “drill-down” primers are primers designed to identify a bioagent at the sub-species level. As used herein, the “sub-species” level of identification includes, but is not limited to, strains, subtypes, variants, and isolates. Drill-down primers are not always required for identification at the sub-species level because broad range survey primers may, in some cases provide sufficient identification resolution to accomplishing this identification objective.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “conserved region” in the context of nucleic acids refers to a nucleobase sequence (e.g., a subsequence of a nucleic acid, etc.) that is the same or similar in two or more different regions or segments of a given nucleic acid molecule (e.g., an intramolecular conserved region), or that is the same or similar in two or more different nucleic acid molecules (e.g., an intermolecular conserved region). To illustrate, a conserved region may be present in two or more different taxonomic ranks (e.g., two or more different genera, two or more different species, two or more different subspecies, and the like) or in two or more different nucleic acid molecules from the same organism. To further illustrate, in certain embodiments, nucleic acids comprising at least one conserved region typically have between about 70%-100%, between about 80-100%, between about 90-100%, between about 95-100%, or between about 99-100% sequence identity in that conserved region. A conserved region may also be selected or identified functionally as a region that permits generation of amplification products via primer extension through hybridization of a completely or partially complementary primer to the conserved region for each of the target sequences to which conserved region is conserved.

The term “correlates” refers to establishing a relationship between two or more things. In certain embodiments, for example, detected molecular masses of one or more amplification products indicate the presence or identity of a given bioagent in a sample. In some embodiments, base compositions are calculated or otherwise determined from the detected molecular masses of amplicons, which base compositions indicate the presence or identity of a given bioagent in a sample.

As used herein, in some embodiments, the term “database” is used to refer to a collection of molecular mass and/or base composition data. The molecular mass and/or base composition data in the database is indexed to bioagents and to primer pairs. The base composition data reported in the database comprises the number of each nucleotide residue in an amplicon defined by each primer pair. The database can also be populated by empirical data determined from amplification products. In this aspect of populating the database, a primer pair is used to generate an amplification product. The molecular mass of the amplification product is determined using a mass spectrometer and the base composition is calculated therefrom without sequencing i.e., without determining the linear sequence of nucleobases comprising the amplification product. It is important to note that amplicon base composition entries in the database are typically derived from sequencing data (i.e., known sequence information), but the base composition of the amplification product being analyzed is determined without sequencing the amplification product. An entry in the database is made to correlate the base composition with the identity of the bioagent and the primer pair used. The database may also be populated using other databases comprising bioagent information. For example, using the GenBank database it is possible to perform electronic PCR using an electronic representation of a primer pair. This in silico method may provide the base composition for any or all selected bioagent(s) stored in the GenBank database. The information may then be used to populate the base composition database as described above. A base composition database can be in silico, a written table, a reference book, a spreadsheet or any form generally amenable to access by data controllers. Preferably, it is in silico on computer readable media.

The term “detect”, “detecting” or “detection” refers to an act of determining the existence or presence of one or more bioagents in a sample.

As used herein, the term “etiology” refers to the causes or origins, of diseases or abnormal physiological conditions.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length sequence or fragment thereof are retained.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more G or T residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (Tₘ) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, “primers” or “primer pairs,” in some embodiments, are oligonucleotides that are designed to bind to conserved sequence regions of one or more bioagent nucleic acids to generate bioagent identifying amplicons. In some embodiments, the bound primers flank an intervening variable region between the conserved binding sequences. Upon amplification, the primer pairs yield amplification products that provide base composition variability between the two or more bioagents. The variability of the base compositions allows for the identification of one or more individual bioagents from, e.g., two or more bioagents based on the base composition distinctions. In some embodiments, the primer pairs are also configured to generate amplification products amenable to molecular mass analysis. Further, the sequences of the primer members of the primer pairs are not necessarily fully complementary to the conserved region of the reference bioagent. For example, in some embodiments, the sequences are designed to be “best fit” amongst a plurality of bioagents at these conserved binding sequences. Therefore, the primer members of the primer pairs have substantial complementarity with the conserved regions of the bioagents, including the reference bioagent.

In some embodiments of the invention, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplification product. When the nucleic acid is double stranded the molecular mass may be determined for either strand or, preferably both strands. In one embodiment, the strands may be separated before introduction into the mass spectrometer, or the strands may be separated by the mass spectrometer itself (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N-6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is used as a term for describing the length of a given segment of nucleic acid and is synonymous with other terms in use in the art including “nucleotide,” “deoxynucleotide,” “nucleotide residue,” and “deoxynucleotide residue.” As is used herein, a nucleobase includes natural and modified nucleotide residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, entitled “Process for preparing polynucleotides” issued Jul. 3, 1984 to Caruthers et al., or other methods known to those skilled in the art. All of these references are incorporated by reference in entirety.

As used herein a “sample” refers to anything capable of being analyzed by the methods provided herein. In some embodiments, the sample comprises or is suspected one or more nucleic acids capable of analysis by the methods. Preferably, the samples comprise nucleic acids (e.g., DNA, RNA, cDNAs, etc.) from one or more strains of Clostridium difficile. Samples can include, for example, urine, feces, rectal swabs, blood, serum/plasma, cerebrospinal fluid (CSF), pleural/synovial/ocular fluids, blood culture bottles, culture isolates, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid. Essentially any sample preparation technique can be utilized to prepare samples for further analysis. In some embodiments, for example, commercially available kits, such as the Ambion TNA kit is optionally utilized.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As is used herein, the term “single primer pair identification” means that one or more bioagents can be identified using a single primer pair. A base composition signature for an amplicon may singly identify one or more bioagents.

As used herein, a “sub-species characteristic” is a genetic characteristic that provides the means to distinguish two members of the same bioagent species. For example, one bacterial strain may be distinguished from another bacterial strain of the same species by possessing a genetic change (e.g., for example, a nucleotide deletion, addition or substitution) in one of the bacterial genes.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved hybridization sequence of a nucleic acid from a given bioagent. Similarly, the primer pairs provided herein may comprise between about 70%-100%, or between about 80-100%, or between about 90-100%, or between about 95-100% identity, or between about 99-100% sequence identity with the primer pairs disclosed in Table 1. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less then 70% sequence homology with a corresponding member of any of the primer pairs of Table 1 if the primer pair has the capability of producing an amplification product corresponding to an amplicon indicating the presence of a strain of Clostridium difficile.

A “system” in the context of analytical instrumentation refers a group of objects and/or devices that form a process line for performing a desired process.

As used herein, “triangulation identification” means the use of more than one primer pair to generate corresponding amplification products for identification of a bioagent. The more than one primer pair can be used in individual wells or vessels or in a multiplex PCR assay. Alternatively, PCR reactions may be carried out in single wells or vessels comprising a different primer pair in each well or vessel. Following amplification the amplification products are pooled into a single well or container which is then subjected to molecular mass analysis. The combination of pooled amplification products can be chosen such that the expected ranges of molecular masses of individual amplification products are not overlapping and thus will not complicate identification of signals. Triangulation is a process of elimination, wherein a first primer pair identifies that an unknown bioagent may be one of a group of bioagents. Subsequent primer pairs are used in triangulation identification to further refine the identity of the bioagent, for example, at the species or sub-species level amongst the subset of possibilities generated with the earlier primer pair. Triangulation identification is complete when the identity of the bioagent at the desired level of identification is determined. The triangulation identification process may also be used to reduce false negative and false positive signals, and enable reconstruction of the origin of hybrid or otherwise engineered bioagents. For example, identification of the three part toxin genes typical of Bacillus anthracis (Bowen et al., J Appl Microbiol., 1999, 87, 270-278) in the absence of the expected compositions from the Bacillus anthracis genome would suggest a genetic engineering event.

As used herein, the term “unknown bioagent” can mean, for example: (i) a bioagent whose existence is not known (for example, the SARS coronavirus was unknown prior to April 2003) and/or (ii) a bioagent whose existence is known (such as the well known bacterial species Staphylococcus aureus for example) but which is not known to be in a sample to be analyzed. For example, if the method for identification of coronaviruses disclosed in commonly owned U.S. patent Ser. No. 10/829,826 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. patent Ser. No. 10/829,826 was employed subsequent to April 2003 to identify the SARS coronavirus in a clinical sample, the second meaning (ii) of “unknown” bioagent would apply because the SARS coronavirus became known to science subsequent to April 2003 because it was not known what bioagent was present in the sample.

As used herein, the term “variable region” is used to describe the intervening region between primer hybridization sites as described herein. The variable region possesses distinct base compositions between at least two bioagents, such that at least one bioagent can be identified at, for example, the family, genus, species or sub-species level. The degree of variability between the at least two bioagents need only be sufficient to allow for identification using mass spectrometry analysis, as described herein.

As used herein, a “wobble base” is a variation in a codon found at the third nucleotide position of a DNA triplet. Variations in conserved regions of sequence are often found at the third nucleotide position due to redundancy in the amino acid code.

Provided herein are methods, compositions, kits, and related systems for the detection and identification of strains of Clostridium difficile.

In some embodiments, primers are selected to hybridize to conserved sequence regions of nucleic acids of strains of Clostridium difficile and which flank variable sequence regions to define a bioagent identifying amplicon. Amplification products corresponding to the amplicon are amenable to molecular mass determination. In some embodiments, the molecular mass is converted to a base composition, which indicates the number of each nucleotide in the amplification product. Systems employing software and hardware useful in converting molecular mass data into base composition information are available from, for example, Ibis Biosciences, Inc. (Carlsbad, Calif.), for example the Ibis T5000 Biosensor System, and are described in U.S. patent application Ser. No. 10/754,415, filed Jan. 9, 2004, incorporated by reference herein in its entirety. In some embodiments, the molecular mass or corresponding base composition of one or more different amplification products is queried against a database of molecular masses or base compositions indexed to bioagents and to the primer pair used to define the amplicon. A match of the measured base composition to a database entry base composition associates the sample bioagent to an indexed bioagent in the database. Thus, the identity of the unknown bioagent is determined. In some instances, the measured base composition associates with more than one database entry base composition. Thus, a second/subsequent primer pair is generally used to generate a second/subsequent amplification product, and its measured base composition is similarly compared to the database to determine its identity in triangulation identification. Furthermore, the methods and other aspects of the invention can be applied to rapid parallel multiplex analyses, the results of which can be employed in a triangulation identification strategy. Thus, in some embodiments, the present invention provides rapid throughput and does not require nucleic acid sequencing or knowledge of the linear sequences of nucleobases of the amplification product for bioagent detection and identification.

Particular embodiments of the mass-spectrum based detection methods are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196; 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338; 2007/0087339; 2007/0087340; 2007/0087341; 2007/0184434; 2007/0218467; 2007/0218467; 2007/0218489; 2007/0224614; 2007/0238116; 2007/0243544; 2007/0248969; 2007/0264661; 2008/0160512; 2008/0311558; 2009/0004643; 2009/0047665; 2009/0125245; WO2002/070664; WO2003/001976; WO2003/100035; WO2004/009849; WO2004/052175; WO2004/053076; WO2004/053141; WO2004/053164; WO2004/060278; WO2004/093644; WO2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222; WO2008/104002; WO2008/116182; WO2008/118809; WO2008/127839; WO2008/143627; WO2008/151023; WO2009/017902; WO2009/023358; WO2009/038840; Ecker et al., Ibis T5000: a universal biosensor approach for microbiology. Nat Rev Microbiol. 2008 Jun. 3; Ecker et al., The Microbial Rosetta Stone Database: A compilation of global and emerging infectious microorganisms and bioterrorist threat agents. BMC Microbiology. 2005. 5(1): 19.; Ecker et al., The Ibis T5000 Universal Biosensor: An Automated Platform for Pathogen Identification and Strain Typing. JALA. 2006. 6(11): 341-351.; Ecker et al., The Microbial Rosetta Stone Database: A common structure for microbial biosecurity threat agents. J Forensic Sci. 2005. 50(6): 1380-5.; Ecker et al., Identification of Acinetobacter species and genotyping of Acinetobacter baumannii by multilocus PCR and mass spectrometry. J Clin Microbiol. 2006 August; 44(8):2921-32.; Ecker et al., Rapid identification and strain-typing of respiratory pathogens for epidemic surveillance. Proc Natl Acad Sci USA. 2005 May 31; 102(22):8012-7. Epub 2005 May 23.; Wortmann et al., Genotypic evolution of Acinetobacter baumannii strains in an outbreak associated with war trauma. Infect Control Hosp Epidemiol. 2008 June; 29(6):553-555.; Hannis et al., High-resolution genotyping of Campylobacter species by use of PCR and high-throughput mass spectrometry. J Clin Microbiol. 2008 April; 46(4):1220-5.; Blyn et al., Rapid detection and molecular serotyping of adenovirus by use of PCR followed by electrospray ionization mass spectrometry. J Clin Microbiol. 2008 February; 46(2):644-51.; Eshoo et al., Direct broad-range detection of alphaviruses in mosquito extracts. Virology. 2007 Nov. 25; 368(2):286-95.; Sampath et al., Global surveillance of emerging Influenza virus genotypes by mass spectrometry. PLoS ONE. 2007 May 30; 2(5):e489.; Sampath et al., Rapid identification of emerging infectious agents using PCR and electrospray ionization mass spectrometry. Ann NY Acad Sci. 2007 April; 1102:109-20.; Hujer et al., Analysis of antibiotic resistance genes in multidrug-resistant Acinetobacter sp. isolates from military and civilian patients treated at the Walter Reed Army Medical Center. Antimicrob Agents Chemother. 2006 December; 50(12):4114-23.; Hall et al., Base composition analysis of human mitochondrial DNA using electrospray ionization mass spectrometry: a novel tool for the identification and differentiation of humans. Anal Biochem. 2005 Sep. 1; 344(1):53-69.; Sampath et al., Rapid identification of emerging pathogens: coronavirus. Emerg Infect Dis. 2005 March; 11(3):373-9.; Jiang Y, Hofstadler S A. A highly efficient and automated method of purifying and desalting PCR products for analysis by electrospray ionization mass spectrometry. Anal Biochem. 2003. 316: 50-57.; Jiang et al., Mitochondrial DNA mutation detection by electrospray mass spectrometry. Clin Chem. 2006. 53(2): 195-203. Epub December 7; Russell et al., Transmission dynamics and prospective environmental sampling of adenovirus in a military recruit setting. J Infect Dis. 2006. 194(7): 877-85. Epub 2006 Aug. 25.; Hofstadler et al., Detection of microbial agents using broad-range PCR with detection by mass spectrometry: The TIGER concept. Chapter in Encyclopedia of Rapid Microbiological Methods. 2006.; Hofstadler et al., Selective ion filtering by digital thresholding: A method to unwind complex ESI-mass spectra and eliminate signals from low molecular weight chemical noise. Anal Chem. 2006. 78(2): 372-378.; Hofstadler et al., TIGER: The Universal Biosensor. Int J Mass Spectrom. 2005. 242(1): 23-41.; Van Ert et al., Mass spectrometry provides accurate characterization of two genetic marker types in Bacillus anthracis. Biotechniques. 2004. 37(4): 642-4, 646, 648.; Sampath et al., Forum on Microbial Threats: Learning from SARS: Preparing for the Next Disease Outbreak—Workshop Summary. (ed. Knobler S E, Mahmoud A, Lemon S.) The National Academies Press, Washington, D.C. 2004. 181-185.

In certain embodiments, amplification products amenable to molecular mass determination produced by the primers described herein are either of a length, size or mass compatible with a particular mode of molecular mass determination, or compatible with a means of providing a fragmentation pattern in order to obtain fragments of a length compatible with a particular mode of molecular mass determination. Such means of providing a fragmentation pattern of an amplification product include, but are not limited to, cleavage with restriction enzymes or cleavage primers, sonication or other means of fragmentation. Thus, in some embodiments, amplification products are larger than 200 nucleobases and are amenable to molecular mass determination following restriction digestion. Methods of using restriction enzymes and cleavage primers are well known to those with ordinary skill in the art.

In some embodiments, amplification products corresponding to bioagent identifying amplicons are obtained using the polymerase chain reaction (PCR). Other amplification methods may be used such as ligase chain reaction (LCR), low-stringency single primer PCR, and multiple strand displacement amplification (MDA). (Michael, S F., Biotechniques (1994), 16:411-412 and Dean et al., Proc Natl Acad. Sci U.S.A. (2002), 99, 5261-5266).

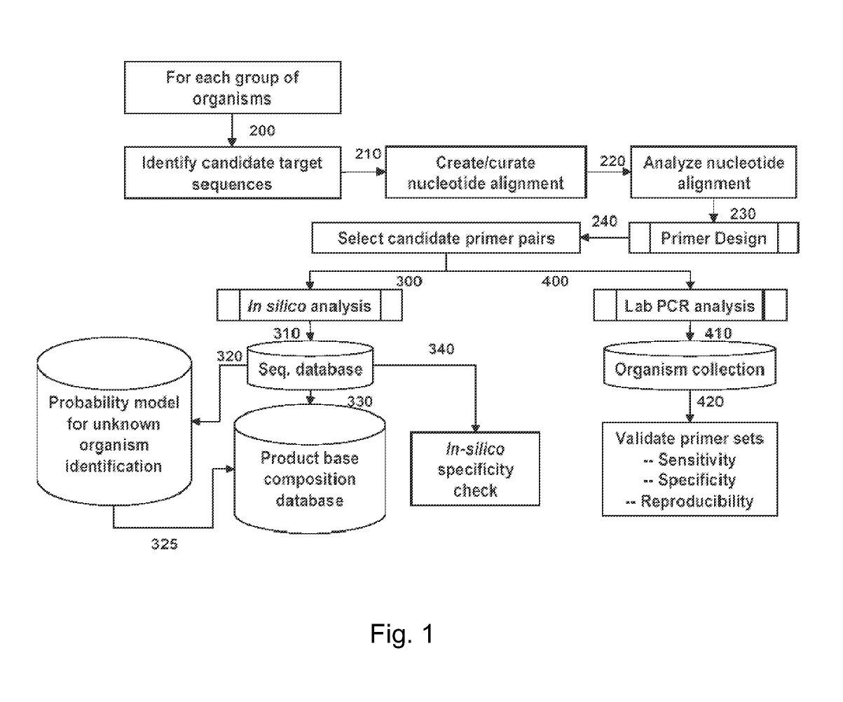

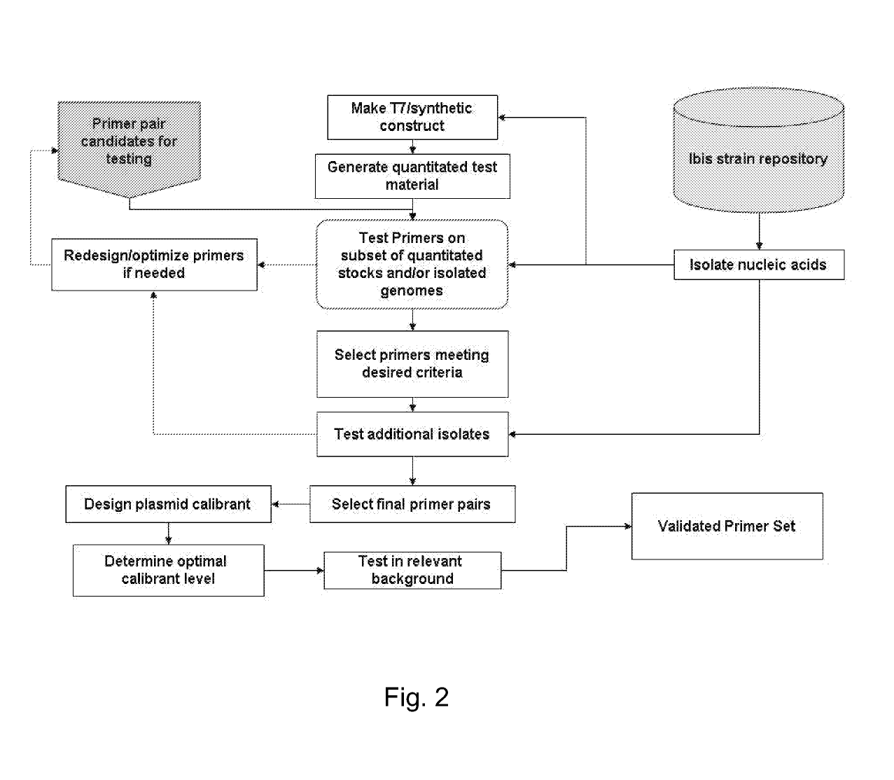

One embodiment of a process flow diagram used for primer selection and validation process is depicted in FIGS. 1 and 2. For each group of organisms, candidate target sequences are identified (200) from which nucleotide sequence alignments are created (210) and analyzed (220). Primers are then configured by selecting priming regions (230) to facilitate the selection of candidate primer pairs (240). Initially, the primer pair sequence is typically a “best fit” amongst the aligned sequences, such that the primer pair sequence may or may not be fully complementary to the hybridization region on any one of the bioagents in the alignment. Thus, best fit primer pair sequences are those with sufficient complementarity with two or more bioagents to hybridize with the two or more bioagents and generate an amplification product. The primer pairs are then subjected to in silico analysis by electronic PCR (ePCR) (300) wherein bioagent identifying amplicons are obtained from sequence databases such as GenBank or other sequence collections (310) and tested for specificity in silico (320). Bioagent identifying amplicons obtained from ePCR of GenBank sequences (310) may also be analyzed by a probability model which predicts the capability of a given amplicon to identify unknown bioagents. Preferably, the base compositions of amplicons with favorable probability scores are then stored in a base composition database (325). Alternatively, base compositions of the bioagent identifying amplicons obtained from the primers and GenBank sequences are directly entered into the base composition database (330). Candidate primer pairs (240) are validated by in vitro amplification by a method such as PCR analysis (400) of nucleic acid from a collection of organisms (410). Amplification products thus obtained are analyzed to confirm the sensitivity, specificity and reproducibility of the primers that define the amplicons (420). If the results of the analysis are not satisfactory, a given primer may be redesigned by lengthening or shortening the primer or changing one or more of the nucleobases of the primer. Such changes may include simple substitution of a nucleobase for one of the remaining three standard nucleobases or by substitution with a modified nucleobase or a universal nucleobase. The skilled person will recognize that the possible solutions to the problem of primer pair redesign is very large and that arriving at any given primer sequence either at the initial “best fit” step or in a subsequent redesign step thus requires significant inventive ingenuity in recognizing why the original primer does not function to a sufficient extent and in choosing a solution to the problem. Much more than routine experimentation is thus required.

Chemical synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers typically are employed as compositions for use in methods for identification of strains of Clostridium difficile as follows: a primer pair composition is contacted with nucleic acid of strains of Clostridium difficile. The nucleic acid is then amplified by a nucleic acid amplification technique, such as PCR for example, to obtain an amplification product that corresponds to a bioagent identifying amplicon. The molecular mass of the strands of the double-stranded amplification product is determined by a molecular mass measurement technique such as mass spectrometry, for example. Preferably the two strands of the double-stranded amplification product are separated during the ionization process. However, they may be separated prior to mass spectrometry measurement. In some embodiments, the mass spectrometer is electrospray Fourier transform ion cyclotron resonance mass spectrometry (ESI-FTICR-MS) or electrospray time of flight mass spectrometry (ESI-TOF-MS). A list of possible base compositions may be generated for the molecular mass value obtained for each strand, and the choice of the base composition from the list is facilitated by matching the base composition of one strand with a complementary base composition of the other strand. A measured molecular mass or base composition calculated therefrom is then compared with a database of molecular masses or base compositions indexed to primer pairs and to known bioagents. A match between the measured molecular mass or base composition of the amplification product and the database-stored molecular mass or base composition for that indexed primer pair correlates the measured molecular mass or base composition with an indexed bioagent, thus identifying the unknown bioagent. In some embodiments, the primer pair used is at least one of the primer pairs of Table 1. In some embodiments, the method is repeated using a different primer pair to resolve possible ambiguities in the identification process or to improve the confidence level for the identification assignment (triangulation identification). In some embodiments, for example, where the unknown is a previously uncharacterized bioagent, the molecular mass or base composition from an amplification product generated from the previously uncharacterized bioagent is matched with one or more best match molecular masses or base compositions from a database to predict a family, genus, species, sub-type, etc. of the previously uncharacterized bioagent. Such information may assist further characterization of the this previously uncharacterized bioagent or provide a physician treating a patient infected by the unknown with a therapeutic agent best calculated to treat the patient.

In certain embodiments, strains of Clostridium difficile are detected with the systems and methods of the present invention in combination with other bioagents, including other viruses, bacteria, fungi, or other bioagents. In particular embodiments, a primer pair panel is employed that includes primer pairs designed for production of amplification products of nucleic acid of strains of Clostridium difficile. Other primer pairs may be included for production of amplification products of bacteria or viruses. Such panels may be specific for a particular type of bioagent, or specific for a specific type of test (e.g., for testing the safety of blood, one may include commonly present viral pathogens such as HCV, HIV, and bacteria that can be contracted via a blood transfusion).

In some embodiments, an amplification product may be produced using only a single primer (either the forward or reverse primer of any given primer pair), provided an appropriate amplification method is chosen, such as, for example, low stringency single primer PCR (LSSP-PCR).

In some embodiments, the oligonucleotide primers are broad range survey primers which hybridize to conserved regions of nucleic acid. The broad range primer may identify the unknown bioagent depending on which bioagent is in the sample. In other cases, the molecular mass or base composition of an amplicon does not provide sufficient resolution to identify the unknown bioagent as any one bioagent at or below the species level. These cases generally benefit from further analysis of one or more amplification products generated from at least one additional broad range survey primer pair, or from at least one additional division-wide primer pair, or from at least one additional drill-down primer pair. Identification of sub-species characteristics may be required, for example, to determine a clinical treatment of patient, or in rapidly responding to an outbreak of a new species, strain, sub-type, etc. of pathogen to prevent an epidemic or pandemic.

One with ordinary skill in the art of design of amplification primers will recognize that a given primer need not hybridize with 100% complementarity in order to effectively prime the synthesis of a complementary nucleic acid strand in an amplification reaction. Primer pair sequences may be a “best fit” amongst the aligned bioagent sequences, thus they need not be fully complementary to the hybridization region of any one of the bioagents in the alignment. Moreover, a primer may hybridize over one or more segments such that intervening or adjacent segments are not involved in the hybridization event (e.g., for example, a loop structure or a hairpin structure). The primers may comprise at least 70%, at least 75%, at least 80%, at least 85%, at least 90%, at least 95% or at least 99% sequence identity with any of the primers listed in Table 1. Thus, in some embodiments, an extent of variation of 70% to 100%, or any range falling within, of the sequence identity is possible relative to the specific primer sequences disclosed herein. To illustrate, determination of sequence identity is described in the following example: a primer 20 nucleobases in length which is identical to another 20 nucleobase primer with the exception of two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 nucleobase primer is completely identical to a 28 nucleobase portion of a 31 nucleobase primer, the 31 nucleobase primer is 90.3% identical to the 28 nucleobase primer (28/31=0.9032).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of nucleic acid is between about 70% and about 80%. In other embodiments, homology, sequence identity or complementarity, is between about 80% and about 90%. In yet other embodiments, homology, sequence identity or complementarity, is at least 90%, at least 92%, at least 94%, at least 95%, at least 96%, at least 97%, at least 98%, at least 99% or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range falling within) sequence identity with the primer sequences specifically disclosed herein.

In some embodiments, the oligonucleotide primers are 14 to 40 nucleobases in length (14 to 40 linked nucleotide residues). These embodiments comprise oligonucleotide primers 14, 15, 16, 17, 18, 19, 20, 21, 22, 23, 24, 25, 26, 27, 28, 29, 30, 31, 32, 33, 34, 35, 36, 37, 38, 39 or 40 nucleobases in length.

In some embodiments, any given primer is provided with a non-templated T residue at the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of, e.g., Taq (Thermophilus aquaticus) DNA polymerase (Magnuson et al., Biotechniques, 1996, 21, 700-709), an occurrence which may lead to ambiguous results arising from molecular mass analysis.

Primers may contain one or more universal bases. Because any variation (due to codon wobble in the third position) in the conserved regions among species is likely to occur in the third position of a DNA (or RNA) triplet, oligonucleotide primers can be designed such that the nucleotide corresponding to this position is a base which can bind to more than one nucleotide, referred to herein as a “universal nucleobase.” For example, under this “wobble” base pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK, an acyclic nucleoside analog containing 5-nitroindazole (Van Aerschot et al., Nucleosides and Nucleotides., 1995, 14, 1053-1056) or the purine analog 1-(2-deoxy-beta-D-ribofuranosyl)-imidazole-4-carboxamide (Sala et al., Nucl Acids Res., 1996, 24, 3302-3306).

In some embodiments, to compensate for weaker binding by the wobble base, oligonucleotide primers are configured such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is incorporated herein by reference in its entirety. Propynylated primers are described in U.S Publication No. 2003/0170682 incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a possible source of ambiguity in the determination of base composition of amplification products. Addition of mass-modifying tags to certain nucleobases of a given primer will result in simplification of de novo determination of base composition of a given amplification product from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹³N and ¹³C.

In some embodiments, the molecular mass of a given amplification product of nucleic acid of strains of Clostridium difficile is determined by mass spectrometry. Mass spectrometry is intrinsically a parallel detection scheme without the need for radioactive or fluorescent labels, because an amplification product is identified by its molecular mass. The current state of the art in mass spectrometry is such that less than femtomole quantities of material can be analyzed to provide information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplification products using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the amplification product. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, assignment of previously unobserved base compositions (also known as “true unknown base compositions”) to a given phylogeny can be accomplished via the use of pattern classifier model algorithms. Base compositions, like sequences, may vary slightly from strain to strain within species, for example. In some embodiments, the pattern classifier model is the mutational probability model. In other embodiments, the pattern classifier is the polytope model. A polytope model is the mutational probability model that incorporates both the restrictions among strains and position dependence of a given nucleobase within a triplet. In certain embodiments, a polytope pattern classifier is used to classify a test or unknown organism according to its amplicon base composition. Such a polytope model is described in PCT Publication No. WO2005089128.

In some embodiments, it is possible to manage this diversity by building “base composition probability clouds” around the composition constraints for each species. A “pseudo four-dimensional plot” may be used to visualize the concept of base composition probability clouds. Optimal primer design typically involves an optimal choice of bioagent identifying amplicons and maximizes the separation between the base composition signatures of individual bioagents. Areas where clouds overlap generally indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of an unknown bioagent whose assigned base composition has not been previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein is bioagent classifying information at a level sufficient to identify a given bioagent. Furthermore, the process of determining a previously unknown base composition for a given bioagent (for example, in a case where sequence information is unavailable) has utility by providing additional bioagent indexing information with which to populate base composition databases. The process of future bioagent identification is thus improved as additional base composition signature indexes become available in base composition databases.

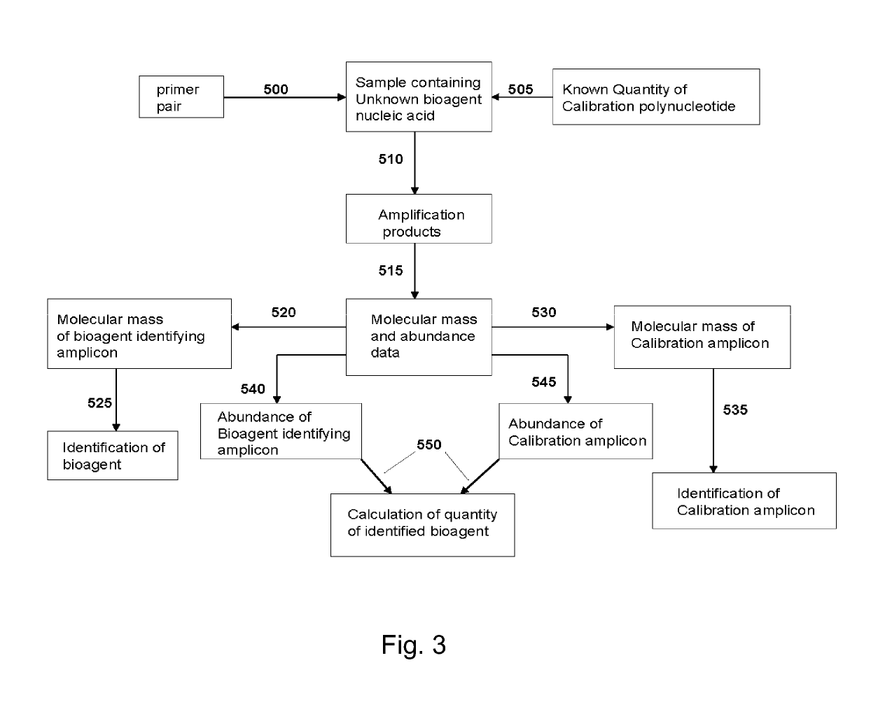

In some embodiments, the identity and quantity of an unknown bioagent may be determined using the process illustrated in FIG. 3. Primers (500) and a known quantity of a calibration polynucleotide (505) are added to a sample containing nucleic acid of an unknown bioagent. The total nucleic acid in the sample is then subjected to an amplification reaction (510) to obtain amplification products. The molecular masses of the amplification products are determined (515) from which are obtained molecular mass and abundance data. The molecular mass of the amplification product corresponding to a bioagent identifying amplicon (520) provides for its identification (525) and the molecular mass of the calibration amplicon obtained from the calibration polynucleotide (530) provides for quantification of the amplification product of the bioagent identifying amplicon (535). The abundance data of the bioagent identifying amplicon is recorded (540) and the abundance data for the calibration data is recorded (545), both of which are used in a calculation (550) which determines the quantity of unknown bioagent in the sample.

In certain embodiments, a sample comprising an unknown bioagent is contacted with a primer pair which amplifies the nucleic acid from the bioagent, and a known quantity of a polynucleotide that comprises a calibration sequence. The amplification reaction then produces two amplification products which correspond to a bioagent identifying amplicon and a calibration amplicon. The amplification products corresponding to the bioagent identifying amplicon and the calibration amplicon are distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent by base composition analysis. The abundance data enables calculation of the quantity of the bioagent, based on the knowledge of the quantity of calibration polynucleotide contacted with the sample.

In some embodiments, construction of a standard curve in which the amount of calibration or calibrant polynucleotide spiked into the sample is varied provides additional resolution and improved confidence for the determination of the quantity of bioagent in the sample. Alternatively, the calibration polynucleotide can be amplified in its own reaction vessel or vessels under the same conditions as the bioagent. A standard curve may be prepared therefrom, and the relative abundance of the bioagent determined by methods such as linear regression. In some embodiments, multiplex amplification is performed where multiple amplification products corresponding to multiple bioagent identifying amplicons are obtained with multiple primer pairs which also amplify the corresponding standard calibration sequences. In this or other embodiments, the standard calibration sequences are optionally included within a single construct (preferably a vector) which functions as the calibration polynucleotide.

In some embodiments, the calibrant polynucleotide is also used as an internal positive control to confirm that amplification conditions and subsequent analysis steps are successful in producing a measurable amplification product. Even in the absence of copies of the genome of a bioagent, the calibration polynucleotide gives rise to an amplification product corresponding to a calibration amplicon. Failure to produce a measurable amplification product corresponding to a calibration amplicon indicates a failure of amplification or subsequent analysis step such as amplicon purification or molecular mass determination. Reaching a conclusion that such failures have occurred is, in itself, a useful event. In other related embodiments, a separate internal positive control polynucleotide may be used. The same strategy used to prepare the calibration polynucleotide may be employed but with an insertion or deletion which is different from the insertion or deletion used in preparation of the internal positive control polynucleotide.

In some embodiments, the calibration sequence is comprised of DNA. In some embodiments, the calibration sequence is comprised of RNA.

In some embodiments, a calibration sequence is inserted into a vector which then functions as the calibration polynucleotide. In some embodiments, more than one calibration sequence is inserted into the vector that functions as the calibration polynucleotide. Such a calibration polynucleotide is herein termed a “combination calibration polynucleotide.” It should be recognized that the calibration method should not be limited to the embodiments described herein. The calibration method can be applied for determination of the quantity of any amplification product corresponding to a bioagent identifying amplicon when an appropriate standard calibrant polynucleotide sequence and/or an appropriate internal positive control polynucleotide are designed and used.

In certain embodiments, primer pairs are configured to produce amplification products corresponding to bioagent identifying amplicons within more conserved regions of nucleic acid of strains of Clostridium difficile. Such regions may evolve quickly and bioagent identifying amplicons corresponding to these regions may be useful for distinguishing emerging strains of Clostridium difficile. Primer pairs that define bioagent identifying amplicons in a conserved region with low probability that the region will evolve past the point of primer recognition are useful, e.g., as a broad range survey-type primer.

The primer pairs described herein provide methods for identifying diseases caused by known or emerging strains of Clostridium difficile. Base composition analysis eliminates the need for prior knowledge of the sequences of these strains for generation of hybridization probes. Thus, in another embodiment, there is provided a method for determining the etiology of a particular disease when the process of identification of is carried out in a clinical setting, and even when a new strain is involved. This is possible because the methods may not be confounded by naturally occurring evolutionary variations.

Another embodiment provides a means of tracking the spread of a given strain of Clostridium difficile when a plurality of samples obtained from different geographical locations are analyzed by methods described above in an epidemiological setting. For example, a plurality of samples from a plurality of different locations may be analyzed with primers which define bioagent identifying amplicons, a subset of which identifies a specific strain. The corresponding locations of the members of the strain-containing subset indicate the spread of the specific strain to the corresponding locations.

Also provided are kits for carrying out the methods described herein. In some embodiments, the kit may comprise a sufficient quantity of one or more primer pairs to perform an amplification reaction on a target polynucleotide from a bioagent which corresponds to a bioagent identifying amplicon. In some embodiments, the kit may comprise from one to twenty primer pairs, from one to ten primer pairs, from one to eight pairs, from one to five primer pairs, from one to three primer pairs, or from one to two primer pairs. In some embodiments, the kit may comprise primer pairs having at least 70% sequence identity with one or more primer pairs recited in Table 1.

In some embodiments, the kit may also comprise a sufficient quantity of a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. In some embodiments, the kit further comprises instructions for analysis, interpretation and dissemination of data acquired by the kit. In other embodiments, instructions for the operation, analysis, interpretation and dissemination of the data of the kit are provided on computer readable media. A kit may also comprise amplification reaction containers such as microcentrifuge tubes, microtiter plates, and the like. A kit may also comprise reagents or other materials for isolating bioagent nucleic acid or amplification products, including, for example, detergents, solvents, or ion exchange resins which may be linked to magnetic beads. A kit may also comprise a table of measured or calculated molecular masses and/or base compositions of bioagents using the primer pairs of the kit.

The invention also provides systems that can be used to perform various assays relating to detection, identification or characterization of strains of Clostridium difficile. In certain embodiments, systems include mass spectrometers configured to detect molecular masses of amplification products produced using purified oligonucleotide primer pairs described herein. Other detectors that are optionally adapted for use in the systems of the invention are described further below. In some embodiments, systems also include controllers operably connected to mass spectrometers and/or other system components. In some of these embodiments, controllers are configured to correlate the molecular masses of the amplification products with the molecular masses of bioagent identifying amplicons of bioagents to effect detection, identification or characterization. In some embodiments, controllers are configured to determine base compositions of the amplification products from the molecular masses of the amplification products. As described herein, the base compositions generally correspond to strains of Clostridium difficile. In certain embodiments, controllers include, or are operably connected to, databases of known molecular masses and/or known base compositions of amplification products of strains of Clostridium difficile produced with the primer pairs described herein. Controllers are described further below.

In some embodiments, systems include one or more of the primer pairs described herein. In certain embodiments, the oligonucleotides are arrayed on solid supports, whereas in others, they are provided in one or more containers, e.g., for assays performed in solution. In certain embodiments, the systems also include at least one detector or detection component (e.g., a spectrometer) that is configured to detect detectable signals produced in the container or on the support. In addition, the systems also optionally include at least one thermal modulator (e.g., a thermal cycling device) operably connected to the containers or solid supports to modulate temperature in the containers or on the solid supports, and/or at least one fluid transfer component (e.g., an automated pipettor) that transfers fluid to and/or from the containers or solid supports, e.g., for performing one or more assays (e.g., nucleic acid amplification, real-time amplicon detection, etc.) in the containers or on the solid supports.

Detectors are typically structured to detect detectable signals produced, e.g., in or proximal to another component of the given assay system (e.g., in a container and/or on a solid support). Suitable signal detectors that are optionally utilized, or adapted for use, herein detect, e.g., fluorescence, phosphorescence, radioactivity, absorbance, refractive index, luminescence, or mass. Detectors optionally monitor one or a plurality of signals from upstream and/or downstream of the performance of, e.g., a given assay step. For example, detectors optionally monitor a plurality of optical signals, which correspond in position to “real-time” results. Example detectors or sensors include photomultiplier tubes, CCD arrays, optical sensors, temperature sensors, pressure sensors, pH sensors, conductivity sensors, or scanning detectors. Detectors are also described in, e.g., Skoog et al., Principles of Instrumental Analysis, 5^{th} Ed., Harcourt Brace College Publishers (1998), Currell, Analytical Instrumentation: Performance Characteristics and Quality, John Wiley & Sons, Inc. (2000), Sharma et al., Introduction to Fluorescence Spectroscopy, John Wiley & Sons, Inc. (1999), Valeur, Molecular Fluorescence: Principles and Applications, John Wiley & Sons, Inc. (2002), and Gore, Spectrophotometry and Spectrofluorimetry: A Practical Approach, 2^{nd} Ed., Oxford University Press (2000), which are each incorporated by reference.

As mentioned above, the systems of the invention also typically include controllers that are operably connected to one or more components (e.g., detectors, databases, thermal modulators, fluid transfer components, robotic material handling devices, and the like) of the given system to control operation of the components. More specifically, controllers are generally included either as separate or integral system components that are utilized, e.g., to receive data from detectors (e.g., molecular masses, etc.), to effect and/or regulate temperature in the containers, or to effect and/or regulate fluid flow to or from selected containers. Controllers and/or other system components are optionally coupled to an appropriately programmed processor, computer, digital device, information appliance, or other logic device (e.g., including an analog to digital or digital to analog converter as needed), which functions to instruct the operation of these instruments in accordance with preprogrammed or user input instructions, receive data and information from these instruments, and interpret, manipulate and report this information to the user. Suitable controllers are generally known in the art and are available from various commercial sources.

Any controller or computer optionally includes a monitor, which is often a cathode ray tube (“CRT”) display, a flat panel display (e.g., active matrix liquid crystal display or liquid crystal display), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. These components are illustrated further below.

The computer typically includes appropriate software for receiving user instructions, either in the form of user input into a set of parameter fields, e.g., in a graphic user interface (GUI), or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming.

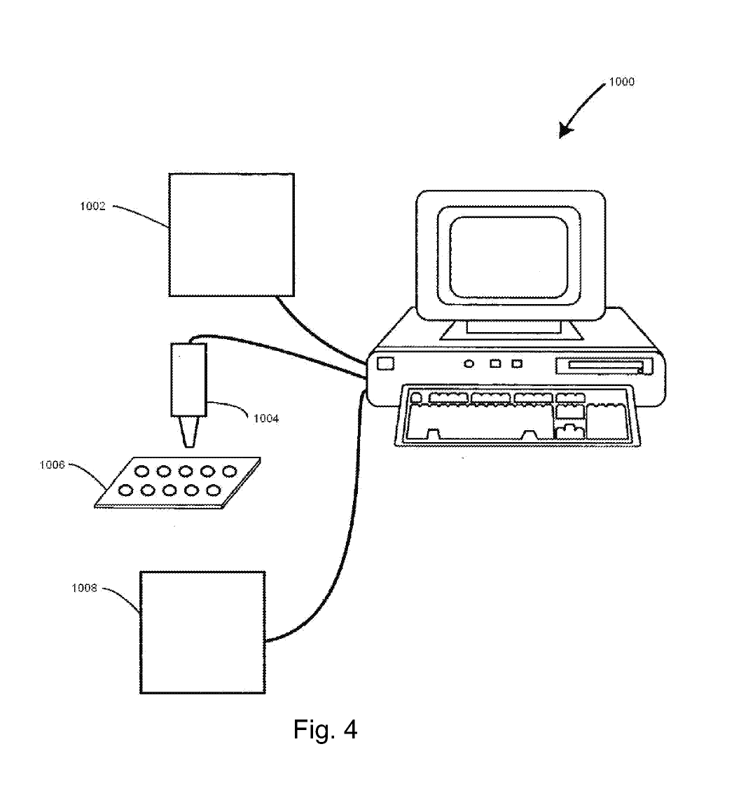

FIG. 4 is a schematic showing a representative system that includes a logic device in which various aspects of the present invention may be embodied. As will be understood by practitioners in the art from the teachings provided herein, aspects of the invention are optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform as desired. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

More specifically, FIG. 4 schematically illustrates computer 1000 to which mass spectrometer 1002 (e.g., an ESI-TOF mass spectrometer, etc.), fluid transfer component 1004 (e.g., an automated mass spectrometer sample injection needle or the like), and database 1008 are operably connected. Optionally, one or more of these components are operably connected to computer 1000 via a server (not shown in FIG. 4). During operation, fluid transfer component 1004 typically transfers reaction mixtures or components thereof (e.g., aliquots comprising amplicons) from multi-well container 1006 to mass spectrometer 1002. Mass spectrometer 1002 then detects molecular masses of the amplicons. Computer 1000 then typically receives this molecular mass data, calculates base compositions from this data, and compares it with entries in database 1008 to identify strains of Clostridium difficile in a given sample. It will be apparent to one of skill in the art that one or more components of the system schematically depicted in FIG. 4 are optionally fabricated integral with one another (e.g., in the same housing).

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

### Example 1

### Design and Validation of Primers that Define Bioagent Identifying Amplicons for Strains of Clostridium difficile

For design of primers that define amplicons for strains of Clostridium difficile, a series of sequences of strains of Clostridium difficile were obtained, aligned and scanned for regions where pairs of PCR primers amplify products of about 29 to about 200 nucleobases in length and distinguish strains of Clostridium difficile by their molecular masses or base compositions. A typical process shown in FIG. 1 is employed for this type of analysis. Primer pair validation is carried out according to some or all of the steps shown in FIG. 2.

A database of expected base compositions for each primer region is generated using an in silico PCR search algorithm, such as (ePCR). An existing RNA structure search algorithm (Macke et al. Nucl. Acids Res., 2001, 29, 4724-4735, incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This also provides information on primer specificity of the selected primer pairs.

Tables 1 to 4 provide information about the primer pairs for identifying strains of Clostridium difficile which are selected according to the processes described above. These tables may be conveniently cross-referenced according to the primer pair number listed in the leftmost column. Table 1 lists the sequences of the forward and reverse primers for each of the primer pairs.

TABLE 1
Sequences of Primer Pairs Designed for
Identification of Strains of Clostridium difficile

| Primer Pair | Primer | Primer | SEQ ID |
|---|---|---|---|
| Number | Direction | Sequence | NO: |
| BCT3749 | Forward | TGCGCAAGACTTA | 18 |
| | | CAAAGCTATAGTGA | |
| BCT3749 | Reverse | TGCTTCTTTTCTTT | 19 |
| | | CCCATTCTTTAGCC | |
| BCT3750 | Forward | TCTGATTGGGAAGA | 8 |
| | | CGAAGATTTGGA | |
| BCT3750 | Reverse | TTGTTCTGCAAAGC | 25 |
| | | TATCTTCCCAC | |
| BCT3751 | Forward | TGTATGGATAGGTG | 21 |
| | | GAGAAGTCAGTG | |
| BCT3751 | Reverse | TACCAAGAATGCTTC | 2 |
| | | ACTATCATACCACAG | |
| BCT3752 | Forward | TGCAGATTGGAGTA | 17 |
| | | TTTAATACACCAGA | |
| BCT3752 | Reverse | TTGATTCTCCCTCAA | 23 |
| | | AATTCTCATCCAAAG | |
| BCT3753 | Forward | TGACCAATTAGCTA | 14 |
| | | GAACACCTGATGA | |
| BCT3753 | Reverse | TCTTATTTGCACCT | 10 |
| | | CATCACCATCTTC | |
| BCT3754 | Forward | TTGGCAAGAAATAACT | 24 |
| | | CAGTAGATGATTTGC | |
| BCT3754 | Reverse | TCCGTTTCTCCCTCTT | 7 |
| | | CATAATGTAAAACTC | |
| BCT3755 | Forward | TGTGCTTATGTGGA | 22 |
| | | TTACCAGTACCTA | |
| BCT3755 | Reverse | TAGCATTCATTTCAT | 5 |
| | | CTGTCATTGCATCTA | |
| BCT3756 | Forward | TACTAAGGGTACAAA | 3 |
| | | TATGAAGATAGGTGC | |
| BCT3756 | Reverse | TGAAACTTCTCCTG | 11 |
| | | TAAATGCTCCTT | |
| BCT3757 | Forward | TGAAGAAAAAGGAG | 13 |
| | | CTTTTACTGGAGA | |
| BCT3757 | Reverse | TCTGTTTCATTGAAG | 9 |
| | | TATTGTCTTCTTTC | |
| BCT3818 | Forward | TGAAAGACGACGAA | 12 |
| | | AAGAGAGCTATTG | |
| BCT3818 | Reverse | TGGCCATAAGTAATA | 20 |
| | | CCAGTATCATATCCT | |
| BCT3898 | Forward | TGAGCACAAAGGAT | 15 |
| | | ATTGCTCTACTGG | |
| BCT3898 | Reverse | TCAGAACAAGCTGGT | 6 |
| | | GAGGATATATTGCC | |
| BCT3899 | Forward | TGAGCACAAAGGGT | 16 |
| | | ATTGCTCTACTGG | |
| BCT3899 | Reverse | TCAGAACAAGCTGGT | 6 |
| | | GAGGATATATTGCC | |
| BCT4265 | Forward | TAAGAGCACAAAGG | 1 |
| | | ATATTGCTCTACTG | |
| BCT4265 | Reverse | TAGAACAAGCTGGT | 4 |
| | | GAGGATATATTGCC | |

Table 2 provides primer pair names constructed of notations which indicate information about the primers and their hybridization coordinates with respect to a reference sequence. The primer pair name “CDTA_AF271719-1-1392_{—}99_{—}222” (primer pair number BCT3749) indicates that the primers of the primer pair are designed to amplify a genome segment in the binary toxin A gene “CDTA” of Clostridium difficile. The reference sequence used in naming the primer pair is of the complete coding sequences of the cdtA and cdtB genes of GenBank Accession No. AF271719. An extraction of residues 1 to 1392 was taken from the sequence of this GenBank accession number. A reference amplicon formed by a theoretical amplification of this extraction with the forward and reverse primers of primer pair no. BCT3749 defines a bioagent identifying amplicon 124 nucleobases in length corresponding to positions 99 to 222 of the extraction of residues 1 to 1392 of GenBank Accession No. AF271719. Thus, with this explanation of the coding of the primer pair names and the additional coding information provided in Tables 3 and 4, a person skilled in the art will understand the coordinates of the amplicons with respect to the reference sequences indicated as well as the exact primer hybridization coordinates. The skilled person will also recognize that while the primer pairs are named with respect to a reference sequence, they are capable of hybridizing to nucleic acid of additional strains of Clostridium difficile for producing amplification products corresponding to bioagent identifying amplicons which may indicate virulence or toxicity of particular strains.

TABLE 2
Primer Pair Name Codes and Reference Amplicon Lengths of Primer
Pairs for Identification of Strains of Clostridium difficile

| | | Reference |
|---|---|---|
| Primer Pair | | Amplicon |
| Number | Primer Pair Name | Length |
| BCT3749 | CDTA_AF271719-1-1392_99_222 | 124 |
| BCT3750 | CDTB_AF271719-1445-4075_634_756 | 123 |
| BCT3751 | TCDA_X92982-12306-20438_297_411 | 115 |
| BCT3752 | TCDB_X92982-3856-10956_6872_6958 | 87 |
| BCT3753 | TCDC_U25132-1036-338_411_513 | 103 |
| BCT3754 | TCDD_X92982-2984-3538_31_137 | 107 |
| BCT3755 | TCDE_X92982-11078-11578_401_487 | 87 |
| BCT3756 | TPI_AY700149-1-449_130_208 | 79 |
| BCT3757 | TPI_AY700149-1-449_175_288 | 114 |
| BCT3818 | TCDC_U25132-1036-338_266_416 | 151 |
| BCT3898 | TCDC_U25132_80_158 | 79 |
| BCT3899 | TCDC_U25132_80_158_2 | 79 |
| BCT4265 | TCDC_U25312_78_157 | 80 |

Table 3 provides names for individual primers of the indicated primer pairs. The individual primer naming convention is similar to that of the primer pairs except that the last two numbered coordinates indicate the hybridization coordinates of the individual primer with respect to the reference sequence whereas the primer pair names indicate the coordinates of the entire amplicon with respect to the reference sequence. For example, the forward primer of primer pair number BCT3749 hybridizes to residues 99 to 125 of an extraction consisting of residues 1 to 1392 of GenBank Accession number AF271719. The final letter code specifies the primer direction, wherein “_F” indicates forward primer and “_R” indicates reverse primer.

TABLE 3
Individual Primer Names of Primer Pairs for Identification of Strains of
Clostridium difficile

| Primer Pair | Primer | |
|---|---|---|
| Number | Direction | Individual Primer Names |
| BCT3749 | Forward | CDTA_AF271719-1-1392_99_125_F |
| BCT3749 | Reverse | CDTA_AF271719-1-1392_195_222_R |
| BCT3750 | Forward | CDTB_AF271719-1445-4075_634_659_F |
| BCT3750 | Reverse | CDTB_AF271719-1445-4075_732_756_R |
| BCT3751 | Forward | TCDA_X92982-12306-20438_297_322_F |
| BCT3751 | Reverse | TCDA_X92982-12306-20438_382_411_R |
| BCT3752 | Forward | TCDB_X92982-3856-10956_6872_6899_F |
| BCT3752 | Reverse | TCDB_X92982-3856-10956_6929_6958_R |
| BCT3753 | Forward | TCDC_U25132-1036-338_411_437_F |
| BCT3753 | Reverse | TCDC_U25132-1036-338_487_513_R |
| BCT3754 | Forward | TCDD_X92982-2984-3538_31_61_F |
| BCT3754 | Reverse | TCDD_X92982-2984-3538_107_137_R |
| BCT3755 | Forward | TCDE_X92982-11078-11578_401_427_F |
| BCT3755 | Reverse | TCDE_X92982-11078-11578_458_487_R |
| BCT3756 | Forward | TPI_AY700149-1-449_130_159_F |
| BCT3756 | Reverse | TPI_AY700149-1-449_183_208_R |
| BCT3757 | Forward | TPI_AY700149-1-449_175_201_F |
| BCT3757 | Reverse | TPI_AY700149-1-449_260_288_R |
| BCT3818 | Forward | TCDC_U25132-1036-338_266_292_F |
| BCT3818 | Reverse | TCDC_U25132-1036-338_387_416_R |
| BCT3898 | Forward | TCDC_U25132_80_106_F |
| BCT3898 | Reverse | TCDC_U25132_130_158_R |
| BCT3899 | Forward | TCDC_U25132_80_106_2_F |
| BCT3899 | Reverse | TCDC_U25132_130_158_R |
| BCT4265 | Forward | TCDC_U25132_78_105_F |
| BCT4265 | Reverse | TCDC_U25132_130_157_R |

Shown in Table 4 are the genome segments which are targeted by the primer pairs.

TABLE 4
Target Genome Segments of Individual Primer Pairs for Identification
of Strains of Clostridium difficile

| | | Reference |
|---|---|---|
| Primer Pair | | Amplicon |
| Number | Target Genome Segment | Length |
| BCT3749 | Binary toxin A | 124 |
| BCT3750 | Binary toxin B | 123 |
| BCT3751 | Toxin A | 115 |
| BCT3752 | Toxin B | 87 |
| BCT3753 | negative regulator of toxin A and B production | 103 |
| | (tcdC) | |
| BCT3754 | Toxin B | 107 |
| BCT3755 | Toxin B | 87 |
| BCT3756 | triosephosphate isomerase gene | 79 |
| BCT3757 | triosephosphate isomerase gene | 114 |
| BCT3818 | negative regulator of toxin A and B production | 151 |
| | (tcdC) | |
| BCT3898 | negative regulator of toxin A and B production | 79 |
| | (tcdC) | |
| BCT3899 | negative regulator of toxin A and B production | 79 |
| | (tcdC) | |
| BCT4265 | negative regulator of toxin A and B production | 80 |
| | (tcdC) | |

### Example 2

### Sample Preparation and PCR

Genomic DNA is prepared from samples using the DNeasy Tissue Kit (Qiagen, Valencia, Calif.) according to the manufacturer's protocols. PCR reactions are typically assembled in 50 μL, reaction volumes in a 96-well microtiter plate format using a Packard MPII liquid handling robotic platform and MJ Dyad® thermocyclers (MJ research, Waltham, Mass.) or Eppendorf Mastercycler thermocyclers (Eppendorf, Westbury, N.Y.). The PCR reaction mixture typically consists of 4 units of Amplitaq Gold, 1× buffer II (Applied Biosystems, Foster City, Calif.), 1.5 mM MgCl₂, 0.4 M betaine, 800 μM dNTP mixture and 250 nM of each primer. The following typical PCR conditions are used: 95° C. for 10 min followed by 8 cycles of 95° C. for 30 seconds, 48° C. for 30 seconds, and 72° C. 30 seconds with the 48° C. annealing temperature increasing 0.9° C. with each of the eight cycles. The PCR is then continued for 37 additional cycles of 95° C. for 15 seconds, 56° C. for 20 seconds, and 72° C. 20 seconds.

### Example 3

### Solution Capture Purification of PCR Products for Mass Spectrometry with Ion Exchange Resin-Magnetic Beads

For solution capture of nucleic acids with ion exchange resin linked to magnetic beads, 25 μL, of a 2.5 mg/mL suspension of BioClone amine-terminated supraparamagnetic beads are added to 25 to 50 μL, of a PCR (or RT-PCR) reaction containing approximately 10 μM of a typical PCR amplification product. This suspension is mixed for approximately 5 minutes by vortexing or pipetting, after which the liquid is removed after using a magnetic separator. The beads containing bound PCR amplification product are then washed three times with 50 mM ammonium bicarbonate/50% MeOH or 100 mM ammonium bicarbonate/50% MeOH, followed by three more washes with 50% MeOH. The bound PCR amplification products are eluted in a solution containing 25 mM piperidine, 25 mM imidazole, 35% MeOH and peptides as mass calibration standards.

### Example 4

### Mass Spectrometry and Base Composition Analysis

The ESI-FTICR mass spectrometer is based on a Bruker Daltonics (Billerica, Mass.) Apex II 70e electrospray ionization Fourier transform ion cyclotron resonance mass spectrometer that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics, can operate in close proximity to the FTICR spectrometer. All aspects of pulse sequence control and data acquisition are performed on a 600 MHz Pentium II data station running Bruker Xmass software under the Windows NT 4.0 operating system. Sample aliquots, typically 15 μL, are extracted directly from 96-well microtiter plates using a CTC HTS PAL autosampler (LEAP Technologies, Carrboro, N.C.) triggered by the FTICR data station. Samples are injected directly into a 10 μL sample loop integrated with a fluidics handling system that supplies the 100 μL/hr flow rate to the ESI source. Ions are formed via electrospray ionization in a modified Analytica (Branford, Conn.) source employing an off axis, grounded electrospray probe positioned approximately 1.5 cm from the metalized terminus of a glass desolvation capillary. The atmospheric pressure end of the glass capillary was biased at 6000 V relative to the ESI needle during data acquisition. A counter-current flow of dry N₂ is employed to assist in the desolvation process. Ions are accumulated in an external ion reservoir comprised of an rf-only hexapole, a skimmer cone, and an auxiliary gate electrode, prior to injection into the trapped ion cell where they are mass analyzed. Ionization duty cycles >99% are achieved by simultaneously accumulating ions in the external ion reservoir during ion detection. Each detection event consists of IM data points digitized over 2.3 s. To improve the signal-to-noise ratio (S/N), 32 scans are co-added for a total data acquisition time of 74 s.

The ESI-TOF mass spectrometer is based on a Bruker Daltonics MicroTOF™. Ions from the ESI source undergo orthogonal ion extraction and are focused in a reflectron prior to detection. The TOF and FTICR are equipped with the same automated sample handling and fluidics described above. Ions are formed in the standard MicroTOF™ ESI source that is equipped with the same off-axis sprayer and glass capillary as the FTICR ESI source. Consequently, source conditions are the same as those described above. External ion accumulation is also employed to improve ionization duty cycle during data acquisition. Each detection event on the TOF mass spectrometer includes 75,000 data points digitized over 75 μs.

The sample delivery scheme allows sample aliquots to be rapidly injected into the electrospray source at high flow rates and to be subsequently electrosprayed at a much lower flow rate for improved ESI sensitivity. Prior to injecting a sample, a bolus of buffer is injected at a high flow rate to rinse the transfer line and spray needle to avoid sample contamination/carryover. Following the rinse step, the autosampler injects the next sample and the flow rate is switched to low flow. Data acquisition begins after a brief equilibration delay. As spectra are co-added, the autosampler continues rinsing the syringe and picking up buffer to rinse the injector and sample transfer line. In general, two syringe rinses and one injector rinse are required to minimize sample carryover. During a routine screening protocol, a new sample mixture is injected every 106 seconds. More recently, a fast wash station for the syringe needle has been implemented which, when combined with shorter acquisition times, facilitates the acquisition of mass spectra at a rate of just under one spectrum/minute.

Raw mass spectra are post-calibrated with an internal mass standard and deconvoluted to monoisotopic molecular masses. Unambiguous base compositions are derived from the exact mass measurements of the complementary single-stranded oligonucleotides. Quantitative results are obtained by comparing the peak heights with an internal PCR calibration standard present in every PCR well at 500 molecules per well. Calibration methods are commonly owned and disclosed in U.S. Patent Application No. 20090004643 which is incorporated herein by reference in entirety.

### Example 5

### De Novo Determination of Base Composition of Amplicons Using Molecular Mass Modified Deoxynucleotide Triphosphates

Because the molecular masses of the four natural nucleobases fall within a narrow molecular mass range (A=313.058, G=329.052, C=289.046, T=304.046, values in Daltons—See, Table 5), a source of ambiguity in assignment of base composition may occur as follows: two nucleic acid strands having different base composition may have a difference of about 1 Da when the base composition difference between the two strands is G⇄A (−15.994) combined with C⇄T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052 is a molecular mass difference of only 0.994 Da. A 1 Da difference in molecular mass may be within the experimental error of a molecular mass measurement and thus, the relatively narrow molecular mass range of the four natural nucleobases imposes an uncertainty factor in this type of situation. One method for removing this theoretical 1 Da uncertainty factor uses amplification of a nucleic acid with one mass-tagged nucleobase and three natural nucleobases.

Addition of significant mass to one of the 4 nucleobases (dNTPs) in an amplification reaction, or in the primers themselves, will result in a significant difference in mass of the resulting amplicon (greater than 1 Da) arising from ambiguities such as the G⇄A combined with C⇄T event (Table 5). Thus, the same G⇄A (−15.994) event combined with 5-Iodo-C⇄T (−110.900) event would result in a molecular mass difference of 126.894 Da. The molecular mass of the base composition A₂₇G₃₀5-Iodo-C₂₁T₂₁ (33422.958) compared with A₂₆G₃₁5-Iodo-C₂₂T₂₀, (33549.852) provides a theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo-C₂₁T₂₁. In contrast, the analogous amplification without the mass tag has 18 possible base compositions.

TABLE 5
Molecular Masses of Natural Nucleobases and the Mass-Modified
Nucleobase 5-Iodo-C and Molecular Mass Differences
Resulting from Transitions

| | Molecular | | Δ Molecular |
|---|---|---|---|
| Nucleobase | Mass | Transition | Mass |
| A | 313.058 | A→T | −9.012 |
| A | 313.058 | A→C | −24.012 |
| A | 313.058 | A→5-Iodo-C | 101.888 |
| A | 313.058 | A→G | 15.994 |
| T | 304.046 | T→A | 9.012 |
| T | 304.046 | T→C | −15.000 |
| T | 304.046 | T→5-Iodo-C | 110.900 |
| T | 304.046 | T→G | 25.006 |
| C | 289.046 | C→A | 24.012 |
| C | 289.046 | C→T | 15.000 |
| C | 289.046 | C→G | 40.006 |
| 5-Iodo-C | 414.946 | 5-Iodo-C→A | −101.888 |
| 5-Iodo-C | 414.946 | 5-Iodo-C→T | −110.900 |
| 5-Iodo-C | 414.946 | 5-Iodo-C→G | −85.894 |
| G | 329.052 | G→A | −15.994 |
| G | 329.052 | G→T | −25.006 |
| G | 329.052 | G→C | −40.006 |
| G | 329.052 | G→5-Iodo-C | 85.894 |

Mass spectra of bioagent-identifying amplicons may be analyzed using a maximum-likelihood processor, as is widely used in radar signal processing. This processor first makes maximum likelihood estimates of the input to the mass spectrometer for each primer by running matched filters for each base composition aggregate on the input data. This includes the response to a calibrant for each primer.

The algorithm emphasizes performance predictions culminating in probability-of-detection versus probability-of-false-detection plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bioagents and may include threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of system noise are factored into this two-stage maximum likelihood calculation. The processor reports the number of molecules of each base composition contained in the spectra. The quantity of amplicon corresponding to the appropriate primer set is reported as well as the quantities of primers remaining upon completion of the amplification reaction.

Base count blurring may be carried out as follows. Electronic PCR can be conducted on nucleotide sequences of the desired bioagents to obtain the different expected base counts that could be obtained for each primer pair. See for example, Schuler, Genome Res. 7:541-50, 1997; or the e-PCR program available from National Center for Biotechnology Information (NCBI, NIH, Bethesda, Md.). In one embodiment one or more spreadsheets from a workbook comprising a plurality of spreadsheets may be used (e.g., Microsoft Excel). First, in this example, there is a worksheet with a name similar to the workbook name; this worksheet contains the raw electronic PCR data. Second, there is a worksheet that contains bioagent name and base count; there is a separate record for each strain after removing sequences that are not identified with a genus and species and removing all sequences for bioagents with less than 10 strains. Third, there is a worksheet that contains the frequency of substitutions, insertions, or deletions for this primer pair. This data is generated by first creating a pivot table from the data worksheet and then executing an Excel VBA macro. The macro creates a table of differences in base counts for bioagents of the same species, but different strains.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by selecting the most abundant strain's base type composition and adding it to the reference set, and then the next most abundant strain's base type composition is added until the threshold is met or exceeded.

For each base count not included in the reference base count set for the bioagent of interest, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260, incorporated herein by reference in entirety.

### Example 6

### Testing of Blinded Clostridium difficile Samples for Identification of Virulence

A series of blinded Clostridium difficile samples were provided by a collaborator. The main objective was to test the primer pairs for the ability to identify virulent strains of Clostridium difficile. These samples were analyzed to determine base compositions of bioagent identifying amplicons defined by primer pair numbers BCT3749, BCT3750, BCT3751, BCT3752, BCT 3756, BCT3757, BCT3818 and BCT 3898. The experimentally-determined base compositions are shown in Tables 6A and 6B. Base compositions corresponding to bioagent identifying amplicons of virulent strains of Clostridium difficile are indicated by bold underlining.

TABLE 6A
Testing of Blinded Clostridium difficile Samples for Identification of Virulence using
Primer Pair Numbers BCT3749, BCT3750, BCT3751 and BCT3752

| | BCT3749 | BCT3750 | BCT3751 | BCT3752 |
|---|---|---|---|---|
| Sample | [A G C T] | [A G C T] | [A G C T] | [A G C T] |
| Not specified | No Detect | No Detect | No Detect | No Detect |
| 1 | 57 25 17 25 | 48 28 13 34 | 40 28 10 37 | 32 18 09 28 |
| 1A | No Detect | No Detect | 40 28 10 37 | 32 18 10 27 |
| 2 | No Detect | No Detect | 40 28 10 37 | 32 18 10 27 |
| 3 | No Detect | No Detect | 40 28 10 37 | 32 18 10 27 |
| 4 | No Detect | No Detect | 40 28 10 37 | 32 18 10 27 |
| 5 | No Detect | No Detect | 40 28 10 37 | 32 18 10 27 |
| 6 | No Detect | No Detect | 40 28 10 37 | 32 18 10 27 |
| not specified | No Detect | No Detect | No Detect | No Detect |
| 7 | No Detect | No Detect | 40 28 10 37 | 32 18 10 27 |
| 8 | No Detect | No Detect | 40 28 10 37 | 32 18 10 27 |
| 9 | 57 25 17 25 | 48 28 13 34 | 40 28 10 37 | 32 18 09 28 |
| 10 | No Detect | No Detect | 40 28 10 37 | 32 18 10 27 |
| 11 | No Detect | No Detect | 40 28 10 37 | 32 18 10 27 |
| 12 | No Detect | No Detect | 40 28 10 37 | 32 18 10 27 |
| 13 | No Detect | No Detect | 40 28 10 37 | 32 18 10 27 |
| not specified | No Detect | No Detect | No Detect | No Detect |
| 14 | No Detect | No Detect | 40 28 10 37 | 32 18 10 27 |
| 15 | No Detect | No Detect | 40 28 10 37 | 32 18 10 27 |
| 16 | No Detect | No Detect | No Detect | No Detect |
| 17 | No Detect | No Detect | No Detect | No Detect |
| 18 | No Detect | No Detect | No Detect | No Detect |
| 20 | No Detect | No Detect | No Detect | No Detect |
| 19 | No Detect | No Detect | No Detect | No Detect |
| not specified | No Detect | No Detect | No Detect | No Detect |
| ATCC_43596 | No Detect | No Detect | 40 28 10 37 | 32 18 10 27 |
| ATCC_43600 | 57 26 14 28 | No Detect | 40 28 10 37 | 32 18 10 27 |
| | (low levels) | | | |
| ATCC_51695 | No Detect | No Detect | 40 28 10 37 | 32 18 10 27 |
| SALINE | No Detect | No Detect | No Detect | No Detect |
| ISOLATION | | | | |
| CONTROL | | | | |
| ATCC_700792 | 57 26 14 28 | No Detect | 40 28 10 37 | 32 18 10 27 |
| | (low levels) | | | |
| ATCC_700057 | 57 26 14 28 | No Detect | No Detect | No Detect |
| | (low levels) | | | |
| ATCC_43598 | No Detect | No Detect | 40 28 10 37 | 32 18 10 27 |
| ATTC_9689 | | | | |
| ATTC_824 | | | | |
| not specified | No Detect | No Detect | No Detect | No Detect |
| II-13 | 57 25 17 25 | 48 28 13 34 | 40 28 10 37 | 32 18 09 28 |
| II-20 | 57 25 17 25 | 48 28 13 34 | 40 28 10 37 | 32 18 09 28 |
| II-27 | 57 25 17 25 | 48 28 13 34 | 40 28 10 37 | 32 18 09 28 |
| II-39 | 57 25 17 25 | 48 28 13 34 | 40 28 10 37 | 32 18 09 28 |
| II-40 | 57 25 17 25 | 48 28 13 34 | 40 28 10 37 | 32 18 09 28 |
| II-46 | 57 25 17 25 | 48 28 13 34 | 40 28 10 37 | 32 18 09 28 |
| II-52 | 57 25 17 25 | 48 28 13 34 | 40 28 10 37 | 32 18 09 28 |
| II-63 | 57 25 17 25 | 48 28 13 34 | 40 28 10 37 | 32 18 09 28 |
| II-79 | 57 25 17 25 | 48 28 13 34 | 40 28 10 37 | 32 18 09 28 |
| II-89 | 57 25 17 25 | 48 28 13 34 | 40 28 10 37 | 32 18 09 28 |
| II-91 | 57 25 17 25 | 48 28 13 34 | 40 28 10 37 | 32 18 09 28 |
| not specified | No Detect | No Detect | No Detect | No Detect |
| II-109 | 57 25 17 25 | 48 28 13 34 | 40 28 10 37 | 32 18 09 28 |
| II-113 | 57 25 17 25 | 48 28 13 34 | 40 28 10 37 | 32 18 09 28 |
| II-204 | 57 25 17 25 | 48 28 13 34 | 40 28 10 37 | 32 18 09 28 |
| II-209 | 57 25 17 25 | 48 28 13 34 | 40 28 10 37 | 32 18 09 28 |
| II-212 | 57 25 17 25 | 48 28 13 34 | 40 28 10 37 | 32 18 09 28 |
| II-116 | No Detect | No Detect | 40 28 10 37 | 32 18 10 27 |
| II-117 | 57 25 17 25 | 48 28 13 34 | 40 28 10 37 | 32 18 09 28 |
| II-118 | 57 25 17 25 | 48 28 13 34 | 40 28 10 37 | 32 18 09 28 |
| II-123 | 57 25 17 25 | 48 28 13 34 | 40 28 10 37 | 32 18 09 28 |
| II-128 | 57 25 17 25 | 48 28 13 34 | 40 28 10 37 | 32 18 09 28 |
| II-136 | 57 25 17 25 | 48 28 13 34 | 40 28 10 37 | 32 18 09 28 |
| not specified | No Detect | No Detect | No Detect | No Detect |
| II-139 | 57 25 17 25 | 48 28 13 34 | 40 28 10 37 | 32 18 09 28 |
| II-140 | 57 25 17 25 | 48 28 13 34 | 40 28 10 37 | 32 18 09 28 |
| II-143 | 57 25 17 25 | 48 28 13 34 | 40 28 10 37 | 32 18 09 28 |
| II-149 | 57 25 17 25 | 48 28 13 34 | 40 28 10 37 | 32 18 09 28 |
| II-153 | 57 25 17 25 | 48 28 13 34 | 40 28 10 37 | 32 18 09 28 |
| II-157 | 57 25 17 25 | 48 28 13 34 | 40 28 10 37 | 32 18 09 28 |
| II-166 | 57 25 17 25 | 48 28 13 34 | 40 28 10 37 | 32 18 09 28 |
| II-168 | 57 25 17 25 | 48 28 13 34 | 40 28 10 37 | 32 18 09 28 |
| II-173 | 57 25 17 25 | 48 28 13 34 | 40 28 10 37 | 32 18 09 28 |
| II-186 | 57 25 17 25 | 48 28 13 34 | 40 28 10 37 | 32 18 09 28 |
| not specified | No Detect | No Detect | No Detect | No Detect |
| II-153 | 57 25 17 25 | 48 28 13 34 | 40 28 10 37 | 32 18 09 28 |
| II-157 | 57 25 17 25 | 48 28 13 34 | 40 28 10 37 | 32 18 09 28 |
| II-166 | 57 25 17 25 | 48 28 13 34 | 40 28 10 37 | 32 18 09 28 |
| II-168 | 57 25 17 25 | 48 28 13 34 | 40 28 10 37 | 32 18 09 28 |
| II-173 | 57 25 17 25 | 48 28 13 34 | 40 28 10 37 | 32 18 09 28 |
| II-186 | 57 25 17 25 | 48 28 13 34 | 40 28 10 37 | 32 18 09 28 |
| II-204 | 57 25 17 25 | 48 28 13 34 | 40 28 10 37 | 32 18 09 28 |
| II-209 | 57 25 17 25 | 48 28 13 34 | 40 28 10 37 | 32 18 09 28 |
| II-212 | 57 25 17 25 | 48 28 13 34 | 40 28 10 37 | 32 18 09 28 |
| not specified | No Detect | No Detect | No Detect | No Detect |
| II-27 | 57 25 17 25 | 48 28 13 34 | 40 28 10 37 | 32 18 09 28 |
| II-116 | 57 25 17 25 | 48 28 13 34 | 40 28 10 37 | 32 18 09 28 |
| II-117 | 57 25 17 25 | 48 28 13 34 | Failed | 32 18 09 28 |
| | | | Reaction | |
| II-118 | 57 25 17 25 | 48 28 13 34 | 40 28 10 37 | 32 18 09 28 |
| II-123 | 57 26 14 28 | No Detect | 40 28 10 37 | 32 18 10 27 |
| | (low levels) | | | |
| II-128 | 57 25 17 25 | 48 28 13 34 | 40 28 10 37 | 32 18 09 28 |
| II-136 | 57 25 17 25 | 48 28 13 34 | 40 28 10 37 | 32 18 09 28 |
| II-139 | 57 25 17 25 | 48 28 13 34 | Failed | 32 18 09 28 |
| | | | Reaction | |
| II-140 | 57 25 17 25 | 48 28 13 34 | 40 28 10 37 | 32 18 09 28 |
| II-143 | 57 25 17 25 | 48 28 13 34 | 40 28 10 37 | 32 18 09 28 |
| II-149 | 57 25 17 25 | 48 28 13 34 | 40 28 10 37 | 32 18 09 28 |

TABLE 6B
Testing of Blinded Clostridium difficile Samples for Identification of Virulence using
Primer Pair Numbers BCT3756, BCT3757, BCT3818 and BCT3898

| | BCT3756 | BCT3757 | BCT3818 | BCT3898 |
|---|---|---|---|---|
| Sample | [A G C T] | [A G C T] | [A G C T] | [A G C T] |
| not specified | No Detect | No Detect | No Detect | No Detect |
| 1 | 33 18 09 19 | 48 23 13 30 | 64 33 15 21 | 17 16 14 31 |
| 1A | 33 18 09 19 | 48 23 13 30 | 73 38 18 22 | 18 16 15 30 |
| 2 | 33 18 09 19 | 48 23 13 30 | 73 38 18 22 | 18 16 15 30 |
| 3 | 33 18 09 19 | 48 23 13 30 | 73 38 18 22 | 18 16 15 30 |
| 4 | 33 18 09 19 | 48 23 13 30 | 73 38 18 22 | 18 16 15 30 |
| 5 | 33 18 09 19 | 48 23 13 30 | 73 38 18 22 | 18 16 15 30 |
| 6 | 33 18 09 19 | 48 23 13 30 | 73 38 18 22 | 18 16 15 30 |
| not specified | No Detect | No Detect | No Detect | No Detect |
| 7 | 33 18 09 19 | 48 23 13 30 | 73 38 18 22 | 18 16 15 30 |
| 8 | 33 18 09 19 | 48 23 13 30 | 73 38 17 23 | single peak |
| 9 | 33 18 09 19 | 48 23 13 30 | 55 30 12 18 | 17 16 14 32 |
| | | | | or 18 16 14 |
| | | | | 31 |
| 10 | 33 18 09 19 | 48 23 13 30 | 73 38 18 22 | 18 16 15 30 |
| 11 | 33 18 09 19 | 48 23 13 30 | 73 38 18 22 | 18 16 15 30 |
| 12 | 33 18 09 19 | 48 23 14 29 | 73 38 18 22 | 18 16 15 30 |
| 13 | 33 18 09 19 | 48 23 13 30 | 73 38 18 22 | 18 16 15 30 |
| not specified | No Detect | No Detect | No Detect | No Detect |
| 14 | 33 18 09 19 | 48 23 13 30 | 73 38 18 22 | 18 16 15 30 |
| 15 | 33 18 09 19 | 48 23 14 29 | 73 38 18 22 | 18 16 15 30 |
| 16 | 33 18 09 19 | 48 23 13 30 | No Detect | No Detect |
| 17 | 33 18 09 19 | 48 23 13 30 | No Detect | No Detect |
| 18 | 33 18 09 19 | 48 23 13 30 | No Detect | No Detect |
| 20 | 33 18 09 19 | 48 23 13 30 | No Detect | No Detect |
| 19 | 33 18 09 19 | 48 23 13 30 | No Detect | No Detect |
| not specified | Failed | No Detect | No Detect | No Detect |
| | Reaction | | | |
| ATCC_43596 | 33 18 09 19 | 48 23 13 30 | 73 38 18 22 | 18 16 15 30 |
| ATCC_43600 | 33 18 09 19 | 48 23 13 30 | 73 38 18 22 | 18 16 15 30 |
| ATCC_51695 | 33 18 09 19 | 48 23 13 30 | 73 38 18 22 | 18 16 15 30 |
| SALINE | No Detect | No Detect | No Detect | No Detect |
| ISOLATION | | | | |
| CONTROL | | | | |
| ATCC_700792 | 33 18 09 19 | 48 23 13 30 | 73 38 18 22 | 18 16 15 30 |
| ATCC_700057 | 33 18 09 19 | 48 23 13 30 | No Detect | No Detect |
| ATCC_43598 | 33 18 09 19 | 48 23 13 30 | 74 37 17 23 | single peak |
| ATTC_9689 | | | 73 38 18 22 | 18 16 15 30 |
| ATTC_824 | | | No Detect | No Detect |
| not specified | No Detect | No Detect | No Detect | No Detect |
| II-13 | 33 18 09 19 | 48 23 13 30 | 64 33 15 21 | 17 16 14 31 |
| II-20 | 33 18 09 19 | 48 23 13 30 | 64 33 15 21 | 17 16 14 31 |
| II-27 | 33 18 09 19 | 48 23 13 30 | 55 30 12 18 | 17 16 14 32 |
| II-39 | 33 18 09 19 | 48 23 13 30 | 64 33 15 21 | 17 16 14 31 |
| II-40 | 33 18 09 19 | 48 23 13 30 | 64 33 15 21 | 17 16 14 31 |
| II-46 | 33 18 09 19 | 48 23 13 30 | 64 33 15 21 | 17 16 14 31 |
| II-52 | 33 18 09 19 | 48 23 13 30 | 64 33 15 21 | 17 16 14 31 |
| II-63 | 33 18 09 19 | 48 23 13 30 | 64 33 15 21 | 17 16 14 31 |
| II-79 | 33 18 09 19 | 48 23 13 30 | 64 33 15 21 | 17 16 14 31 |
| II-89 | 33 18 09 19 | 48 23 13 30 | 64 33 15 21 | 17 16 14 31 |
| II-91 | 33 18 09 19 | 48 23 13 30 | 64 33 15 21 | 17 16 14 31 |
| not specified | No Detect | No Detect | No Detect | No Detect |
| II-109 | 33 18 09 19 | 48 23 13 30 | 64 33 15 21 | 17 16 14 31 |
| II-113 | 33 18 09 19 | 48 23 13 30 | 64 33 15 21 | 17 16 14 31 |
| II-204 | 33 18 09 19 | 48 23 13 30 | 64 33 15 21 | 17 16 14 31 |
| II-209 | 33 18 09 19 | 48 23 13 30 | 64 33 15 21 | 17 16 14 31 |
| II-212 | 33 18 09 19 | 48 23 13 30 | 64 33 15 21 | 17 16 14 31 |
| II-116 | 33 18 09 19 | 48 23 13 30 | 63 34 15 21 | single peak |
| II-117 | 33 18 09 19 | 48 23 13 30 | 64 33 15 21 | 17 16 14 31 |
| II-118 | 33 18 09 19 | 48 23 13 30 | 64 33 15 21 | 17 16 14 31 |
| II-123 | 33 18 09 19 | 48 23 13 30 | 64 33 15 21 | 17 16 14 31 |
| II-128 | 33 18 09 19 | 48 23 13 30 | 64 33 15 21 | 17 16 14 31 |
| II-136 | 33 18 09 19 | 48 23 13 30 | 64 33 15 21 | 17 16 14 31 |
| not specified | No Detect | No Detect | No Detect | No Detect |
| II-139 | 33 18 09 19 | 48 23 13 30 | 64 33 15 21 | 17 16 14 31 |
| II-140 | 33 18 09 19 | 48 23 13 30 | 64 33 15 21 | 17 16 14 31 |
| II-143 | 33 18 09 19 | 48 23 13 30 | 64 33 15 21 | 17 16 14 31 |
| II-149 | 33 18 09 19 | 48 23 13 30 | 73 38 17 23 | single peak |
| II-153 | 33 18 09 19 | 48 23 13 30 | 64 33 15 21 | 17 16 14 31 |
| II-157 | 33 18 09 19 | 48 23 13 30 | 55 30 12 18 | 17 16 14 32 |
| II-166 | 33 18 09 19 | 48 23 13 30 | 74 37 17 23 | single peak |
| II-168 | 33 18 09 19 | 48 23 13 30 | 64 33 15 21 | 17 16 14 31 |
| II-173 | 33 18 09 19 | 48 23 13 30 | 64 33 15 21 | 17 16 14 31 |
| II-186 | 33 18 09 19 | 48 23 13 30 | 64 33 15 21 | 17 16 14 31 |
| not specified | No Detect | No Detect | No Detect | No Detect |
| II-153 | 33 18 09 19 | 48 23 13 30 | 64 33 15 21 | 17 16 14 31 |
| II-157 | 33 18 09 19 | 48 23 13 30 | 64 33 15 21 | 17 16 14 31 |
| II-166 | 33 18 09 19 | 48 23 13 30 | 73 38 17 23 | single peak |
| II-168 | 33 18 09 19 | 48 23 13 30 | 64 33 15 21 | 17 16 14 31 |
| II-173 | 33 18 09 19 | 48 23 13 30 | 55 30 12 18 | 17 16 14 32 |
| II-186 | 33 18 09 19 | 48 23 13 30 | 74 37 17 23 | single peak |
| II-204 | 33 18 09 19 | 48 23 13 30 | 64 33 15 21 | 17 16 14 31 |
| II-209 | 33 18 09 19 | 48 23 13 30 | 64 33 15 21 | 17 16 14 31 |
| II-212 | 33 18 09 19 | 48 23 13 30 | 64 33 15 21 | 17 16 14 31 |
| not specified | No Detect | No Detect | No Detect | Failed |
| | | | | Reaction |
| II-27 | 33 18 09 19 | 48 23 13 30 | 55 30 12 18 | 17 16 14 32 + |
| | | | | 18 16 14 31 |
| II-116 | 33 18 09 19 | 48 23 13 30 | 64 33 15 21 | 17 16 14 31 |
| II-117 | 33 18 09 19 | 48 23 13 30 | 64 33 15 21 | 17 16 14 31 |
| II-118 | 33 18 09 19 | 48 23 13 30 | 64 33 15 21 | 17 16 14 31 |
| II-123 | 33 18 09 19 | 48 23 13 30 | 63 34 15 21 | single peak |
| II-128 | 33 18 09 19 | 48 23 13 30 | 64 33 15 21 | 17 16 14 31 |
| II-136 | 33 18 09 19 | 48 23 13 30 | 64 33 15 21 | 17 16 14 31 |
| II-139 | 33 18 09 19 | 48 23 13 30 | 64 33 15 21 | 17 16 14 31 |
| II-140 | 33 18 09 19 | 48 23 13 30 | 64 33 15 21 | 17 16 14 31 |
| II-143 | 33 18 09 19 | 48 23 13 30 | 64 33 15 21 | 17 16 14 31 |
| II-149 | 33 18 09 19 | 48 23 13 30 | 64 33 15 21 | 17 16 14 31 |

The results of Table 6A and 6B indicate that samples 1, II-13, II-20, II-39, II-40, II-46, II-52, II-63, II-79, II-89, II-91, II-109, II-113, II-204, II-209, II-212, II-117, II-118, II-123, II-128, II-136, II-139, II-140, II-143, and II-153 represent virulent strains. Primer pair BCT3818 is thus demonstrated to be particularly useful by identifying the virulent NAP-1 strain which has a truncated form of the tcdC gene due to an 18 nucleobase deletion. Detection of the virulent strain is also confirmed by primer pair number BCT3898 which indicates a single nucleobase deletion. Primer pair number BCT3752 identifies the presence of the toxin B gene which was also present in all of the NAP-1-identified samples indicated above. Samples 9, II-27, II-149, II-166 and II-186 were found to contain the toxin B gene but not the truncated tcdC gene. The remaining primer pairs produced amplification products which confirmed that the samples contain Clostridium difficile and are expected to be useful in future analyses where newly emergent strains are identified.

Table 7 shows a summary of the results of detections of Toxin B, Binary Toxin and the tcdC deletion obtained in the analysis above. The unblended strain types are included.

TABLE 7
Summary of Results of Detections of Virulence Markers in
Selected Samples

| | | | | Strain |
|---|---|---|---|---|
| | | Binary | TcdC | type |
| Sample | Toxin B | toxin | Deletion | (PFGE) |
| II-13 | + | + | + | Nap1 |
| II-20 | + | + | + | Nap1 |
| II-27 | + | + | + | IX |
| II-39 | + | + | + | Nap1 |
| II-40 | + | + | + | Nap1a |
| II-46 | + | + | + | Nap1a |
| II-52 | + | + | + | NAP1 |
| II-63 | + | + | + | Nap1a |
| II-79 | + | + | + | Nap1a |
| II-89 | + | + | + | Nap1a |
| II-91 | + | + | + | NAP1 |
| II-109 | + | + | + | Nap1 |
| II-113 | + | + | + | Nap1a |
| II-204 | + | + | + | Nap1a |
| II-209 | + | + | + | Nap1 |
| II-212 | + | + | + | Nap1a |
| II-116 | + | + | + | Nap1a |
| II-117 | + | + | + | Nap1a |
| II-118 | + | + | + | Nap1a |
| II-123 | + | + | + | VIII |
| II-128 | + | + | + | Nap1a |
| II-136 | + | + | + | Nap1a |
| II-139 | + | + | + | Nap1a |
| II-140 | + | + | + | Nap1a |
| II-143 | + | + | + | Nap1 |
| II-149 | + | + | − | Nap1a |
| II-153 | + | + | + | Nap1a |
| II-157 | + | + | + | Nap1a |
| II-166 | + | + | − | VII |
| II-168 | + | + | + | Nap1a |
| II-173 | + | + | + | IX |
| II-186 | + | + | − | VII |

In most cases, the strain features identified by base composition analysis matched the known strain features of the known strains of Clostridium difficile. This indicates that the primer pairs used for identifying the strain features are operating as intended.

### Example 7

### Testing of Selected Samples with a Primer Pair Panel Including Redesigned Primer Pair BCT4265

A selected group of samples from the sample series presented in Example 6 was tested again using a revised primer pair panel which also included primer pair number BCT4265. The results are shown in Tables 8A and 8B. Base compositions corresponding to bioagent identifying amplicons of virulent strains of Clostridium difficile are indicated by bold underlining.

TABLE 8A
Testing of Blinded Clostridium difficile Samples for Identification of Virulence
using Primer Pair Numbers BCT3749, BCT3750, BCT3751 and BCT3752

| | BCT3749 | BCT3750 | BCT3751 | BCT3752 |
|---|---|---|---|---|
| Sample | [A G C T] | [A G C T] | [A G C T] | [A G C T] |
| 1 | 57 25 17 25 | 48 28 13 34 | 40 28 10 37 | 32 18 09 28 |
| 9 | 57 25 17 25 | 48 28 13 34 | 40 28 10 37 | 32 18 09 28 |
| ATCC_43600 | No Detect | No Detect | 40 28 10 37 | 32 18 10 27 |
| II-27 | 57 25 17 25 | 48 28 13 34 | 40 28 10 37 | 32 18 09 28 |
| II-149 | 57 25 17 25 | 48 28 13 34 | 40 28 10 37 | 32 18 09 28 |
| II-166 | 57 25 17 25 | 48 28 13 34 | 40 28 10 37 | 32 18 09 28 |
| ATCC_43598 | No Detect | No Detect | 40 28 10 37 | 32 18 10 27 |
| II-27 | 57 25 17 25 | 48 28 13 34 | 40 28 10 37 | 32 18 09 28 |
| II-109 | 57 25 17 25 | 48 28 13 34 | 40 28 10 37 | 32 18 09 28 |
| II-113 | 57 25 17 25 | 48 28 13 34 | 40 28 10 37 | 32 18 09 28 |
| II-149 | 57 25 17 25 | 48 28 13 34 | 40 28 10 37 | 32 18 09 28 |
| II-166 | 57 25 17 25 | 48 28 13 34 | 40 28 10 37 | 32 18 09 28 |

TABLE 8B
Testing of Blinded Clostridium difficile Samples for Identification of Virulence
using Primer Pair Numbers BCT3756, BCT3756, BCT3818 and BCT4265

| | BCT3756 | BCT3757 | BCT3818 | BCT4265 |
|---|---|---|---|---|
| Sample | [A G C T] | [A G C T] | [A G C T] | [A G C T] |
| 33 18 09 19 | Failed | 64 33 15 21 | 19 15 14 31 | 33 18 09 19 |
| | Reaction | | | |
| 33 18 09 19 | 48 23 13 30 | 55 30 12 18 | 19 15 14 32 | 33 18 09 19 |
| 33 18 09 19 | 48 23 13 30 | 73 38 18 22 | 20 15 15 30 | 33 18 09 19 |
| 33 18 09 19 | 48 23 13 30 | 55 30 12 18 | 19 15 14 32 | 33 18 09 19 |
| 33 18 09 19 | 48 23 13 30 | 64 33 15 21 | 19 15 14 31 | 33 18 09 19 |
| 33 18 09 19 | 48 23 13 30 | 73 38 17 23 | 19 16 15 30 | 33 18 09 19 |
| 33 18 09 19 | 48 23 13 30 | 74 37 17 23 | 20 15 14 31 | 33 18 09 19 |
| 33 18 09 19 | 48 23 13 30 | 55 30 12 18 | 19 15 14 32 | 33 18 09 19 |
| 33 18 09 19 | 48 23 13 30 | 64 33 15 21 | 19 15 14 31 | 33 18 09 19 |
| 33 18 09 19 | 48 23 13 30 | 64 33 15 21 | 19 15 14 31 | 33 18 09 19 |
| 33 18 09 19 | 48 23 13 30 | 64 33 15 21 | 19 15 14 31 | 33 18 09 19 |
| 33 18 09 19 | 48 23 13 30 | 73 38 17 23 | 19 16 15 30 | 33 18 09 19 |

It can be seen that the base compositions of amplification products produced by primer pair BCT4265 for each of these samples were identical and thus do not provide resolving power. It was decided that primer pair number BCT4265 would not be further investigated.

### Example 8

### Selection of a Panel of Primer Pairs for Future Analyses of Clostridium difficile

The results of the foregoing analyses as well as testing of primer pairs for the ability to amplify low levels of calibrant polynucleotides and internal positive control polynucleotides (not shown) has led to the selection of a panel of eight primer pairs as shown in Table 9.

TABLE 9
Primer Pair Name Codes and Reference Amplicon Lengths of
Primer Pairs for an Eight Primer Pair Panel for Identification of
Strains of Clostridium difficile

| | | Reference |
|---|---|---|
| Primer Pair | | Amplicon |
| Number | Primer Pair Name | Length |
| BCT3749 | CDTA_AF271719-1-1392_99_222 | 124 |
| BCT3750 | CDTB_AF271719-1445-4075_634_756 | 123 |
| BCT3751 | TCDA_X92982-12306-20438_297_411 | 115 |
| BCT3755 | TCDE_X92982-11078-11578_401_487 | 87 |
| BCT3756 | TPI_AY700149-1-449_130_208 | 79 |
| BCT3757 | TPI_AY700149-1-449_175_288 | 114 |
| BCT3818 | TCDC_U25132-1036-338_266_416 | 151 |
| BCT3898 | TCDC_U25132_80_158 | 79 |

The function of each of the primer pairs of Table 9 is outlined in Table 10.

| Primer Pair | Role of Each Primer Pair in Determining Strain |
|---|---|
| Number | Features in Clostridium difficile |
| BCT3749 | Determination of Binary toxin cdtA and cdtB. May |
| | indicated virulence |
| BCT3750 | Determination of Binary toxin cdtA and cdtB. May |
| | indicated virulence |
| BCT3751 | Determination of presence of Toxin A |
| BCT3755 | Determination of presence of toxin B |
| BCT3756 | Broad detection of Clostridium difficile |
| BCT3757 | Broad detection of Clostridium difficile (does not |
| | differentiate from Clostridium tetani) |
| BCT3818 | tcdC 18 nucleobase deletion determines virulence |
| BCT3898 | Single bp deletion marks virulence |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

We claim:

1. A purified oligonucleotide primer pair for identifying a strain of Clostridium difficile in a sample, said primer pair comprising a forward primer and a reverse primer, each configured to hybridize to nucleic acid of two or more different species or strains of bacteria in a nucleic acid amplification reaction which produces an amplification product between about 29 to about 200 nucleobases in length, said amplification product comprising portions corresponding to a forward primer hybridization region, a reverse primer hybridization region and an intervening region having a base composition which varies among amplification products produced from nucleic acid of said two or more different species or strains of bacteria, said base composition of said intervening region providing a means for identifying said a strain of Clostridium difficile.

2. The primer pair of claim 1 wherein said strain of Clostridium difficile comprises a subspecies characteristic selected from the group consisting of: binary toxin cdtA, and binary toxin cdtB, toxin A, toxin B, an 18-nucleobase 18 nucleobase deletion within the tcdC gene, and a single nucleobase deletion within the tcdC gene.

3. The primer pair of claim 2 wherein each member of said primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

4. The primer pair of claim 3 wherein said forward primer and said reverse primer are about 14 to about 40 nucleobases in length.

5. The primer pair of claim 3, wherein said forward primer or said reverse primer or both further comprise a non-templated thymidine residue on the 5′-end.

6. The primer pair of claim 3, wherein said forward primer or said reverse primer or both further comprise at least one molecular mass modifying tag.

7. The primer pair of claim 3, wherein said forward primer or said reverse primer or both further comprise at least one modified nucleobase.

8. The primer pair of claim 7, wherein said modified nucleobase is 5-propynyluracil or 5-propynylcytosine.

9. The primer pair of claim 7, wherein said modified nucleobase is a mass-modified nucleobase.

10. The primer pair of claim 9, wherein said mass-modified nucleobase is 5-iodo-cytosine.

11. The primer pair of claim 7, wherein said modified nucleobase is a universal nucleobase.

12. The primer pair of claim 11, wherein said universal nucleobase is inosine.

13. The primer pair of claim 1 wherein said strain of Clostridium difficile is selected from the group consisting of Clostridium difficile Nap1, Clostridium difficile Nap1a, Clostridium difficile IX, Clostridium difficile VII and Clostridium difficile VIII.

14. An isolated amplification product for identification of strain of Clostridium difficile, said amplification product produced by a process comprising:
a) amplifying nucleic acid of a bacterium in a reaction mixture comprising a primer pair, said primer pair comprising a forward primer and a reverse primer, each configured to hybridize to nucleic acid of two or more different strains of Clostridium difficile in a nucleic acid amplification reaction, said amplification product having a length of about 29 to about 200 nucleobases and comprising portions corresponding to a forward primer hybridization region, a reverse primer hybridization region and an intervening region having a base composition which varies among amplification products produced from nucleic acid of said two or more different strains of Clostridium difficile, said base composition of said intervening region providing a means for identifying said strain of Clostridium difficile; and
b) isolating said amplification product from said reaction mixture.

15. The amplification product of claim 14 wherein said isolating step is performed using an anion exchange resin linked to a magnetic bead.

16. The amplification product of claim 14 wherein each member of said primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

17. The amplification product of claim 16 wherein said forward primer and said reverse primer are about 14 to about 40 nucleobases in length.

18. The amplification product of claim 16, wherein said forward primer or said reverse primer or both further comprise a non-templated thymidine residue on the 5′-end.

19. The amplification product of claim 16, wherein said forward primer or said reverse primer or both further comprise at least one molecular mass modifying tag.

20. The amplification product of claim 16, wherein said forward primer or said reverse primer or both further comprise at least one modified nucleobase.

21. The amplification product of claim 20, wherein said modified nucleobase is 5-propynyluracil or 5-propynylcytosine.

22. The amplification product of claim 20, wherein said modified nucleobase is a mass-modified nucleobase.

23. The amplification product of claim 22, wherein said mass-modified nucleobase is 5-iodo-cytosine.

24. The amplification product of claim 22, wherein said modified nucleobase is a universal nucleobase.

25. The amplification product of claim 24, wherein said universal nucleobase is inosine.

26. A method for identifying a strain of Clostridium difficile in a sample said method comprising:
(a) obtaining an amplification product by amplifying nucleic acid of a bacterium in said sample using the primer pair of claim 1;
(b) measuring the molecular mass of one or both strands of said amplification product;
(c) comparing said molecular mass to a plurality of database-stored molecular masses of strands of amplification products of known strains of Clostridium difficile; and
d) identifying a match between said molecular mass and at least one of said database-stored molecular masses of amplification products, thereby identifying said strain of Clostridium difficile.

27. The method of claim 26 wherein each member of said primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

28. The method of claim 27 wherein said nucleic acid comprises a subspecies characteristic selected from the group consisting of: binary toxin cdtA, and binary toxin cdtB, toxin A, toxin B, an 18-nucleobase 18 nucleobase deletion within the tcdC gene, and a single nucleobase deletion within the tcdC gene.

29. The method of claim 26 wherein said molecular mass is determined by mass spectrometry.

30. A method for identifying a strain of Clostridium difficile in a sample, said method comprising:
(a) obtaining an amplification product by amplifying nucleic acid of a strain of Clostridium difficile in said sample using the purified primer pair of claim 1;
(b) measuring the molecular mass of one or both strands of said amplification product;
(c) determining the base composition of said amplification product from said molecular mass;
(d) comparing said base composition to a plurality of database-stored base compositions of strands of amplification products of known strains of Clostridium difficile; and
(e) identifying a match between said base composition and at least one of said database-stored molecular masses of amplification products, thereby identifying said strain of Clostridium difficile.

31. The method of claim 30 wherein each member of said primer pair has at least 70% sequence identity with a corresponding member of a primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.

32. The method of claim 31 wherein said nucleic acid comprises a subspecies characteristic selected from the group consisting of: binary toxin cdtA, and binary toxin cdtB, toxin A, toxin B, an 18-nucleobase 18 nucleobase deletion within the tcdC gene, and a single nucleobase deletion within the tcdC gene.

33. The method of claim 30 wherein said molecular mass is determined by mass spectrometry.

34. A kit comprising one or more purified primer pairs for identifying a strain of Clostridium difficile in a sample, each member of said one or more primer pairs having at least 70% sequence identity with a corresponding member of one or more primer pairs selected from the group consisting of: SEQ ID NOs: 18:19, 8:25, 21:2, 17:23, 14:10, 24:7, 22:5, 3:11, 13:9, 12:20, 15:6, 16:6 and 1:4.

35. The kit of claim 34 further comprising deoxynucleotide triphosphates.

36. The kit of claim 34 wherein one or more of said deoxynucleotide triphosphates is 13C-enriched.

37. A system, comprising:
(a) a mass spectrometer configured to detect one or more molecular masses of an amplification product of claim 14;
(b) a database of known molecular masses and/or known base compositions of amplification products of strains of Clostridium difficile; and
(b) a controller operably connected to said mass spectrometer and to said database said controller configured to match said molecular masses of said amplification product with a measured or calculated molecular mass of a corresponding amplification product of a strain of Clostridium difficile.

38. The system of claim 47 wherein said database of known molecular masses and/or known base compositions of amplification products of strains of Clostridium difficile includes amplification products defined by one or more primer pairs wherein each member of said one or more primer pairs has at least 70% sequence identity with a corresponding member of a corresponding primer pair selected from the group consisting of: SEQ ID NOs: 18:19, 8:25, 21:2, 17:23, 14:10, 24:7, 22:5, 3:11, 13:9, 12:20, 15:6, 16:6 and 1:4.


SEQUENCE LISTING (25 sequences)

SEQ ID NO: 1 - DNA, 28 bp, Artificial Sequence
  Primer
taagagcacaaaggatattgctctactg

SEQ ID NO: 2 - DNA, 30 bp, Artificial Sequence
  Primer
taccaagaatgcttcactatcataccacag

SEQ ID NO: 3 - DNA, 30 bp, Artificial Sequence
  Primer
tactaagggtacaaatatgaagataggtgc

SEQ ID NO: 4 - DNA, 28 bp, Artificial Sequence
  Primer
tagaacaagctggtgaggatatattgcc

SEQ ID NO: 5 - DNA, 30 bp, Artificial Sequence
  Primer
tagcattcatttcatctgtcattgcatcta

SEQ ID NO: 6 - DNA, 29 bp, Artificial Sequence
  Primer
tcagaacaagctggtgaggatatattgcc

SEQ ID NO: 7 - DNA, 31 bp, Artificial Sequence
  Primer
tccgtttctccctcttcataatgtaaaactc

SEQ ID NO: 8 - DNA, 26 bp, Artificial Sequence
  Primer
tctgattgggaagacgaagatttgga

SEQ ID NO: 9 - DNA, 29 bp, Artificial Sequence
  Primer
tctgtttcattgaagtattgtcttctttc

SEQ ID NO: 10 - DNA, 27 bp, Artificial Sequence
  Primer
tcttatttgcacctcatcaccatcttc

SEQ ID NO: 11 - DNA, 26 bp, Artificial Sequence
  Primer
tgaaacttctcctgtaaatgctcctt

SEQ ID NO: 12 - DNA, 27 bp, Artificial Sequence
  Primer
tgaaagacgacgaaaagagagctattg

SEQ ID NO: 13 - DNA, 27 bp, Artificial Sequence
  Primer
tgaagaaaaaggagcttttactggaga

SEQ ID NO: 14 - DNA, 27 bp, Artificial Sequence
  Primer
tgaccaattagctagaacacctgatga

SEQ ID NO: 15 - DNA, 27 bp, Artificial Sequence
  Primer
tgagcacaaaggatattgctctactgg

SEQ ID NO: 16 - DNA, 27 bp, Artificial Sequence
  Primer
tgagcacaaagggtattgctctactgg

SEQ ID NO: 17 - DNA, 28 bp, Artificial Sequence
  Primer
tgcagattggagtatttaatacaccaga

SEQ ID NO: 18 - DNA, 27 bp, Artificial Sequence
  Primer
tgcgcaagacttacaaagctatagtga

SEQ ID NO: 19 - DNA, 28 bp, Artificial Sequence
  Primer
tgcttcttttctttcccattctttagcc

SEQ ID NO: 20 - DNA, 30 bp, Artificial Sequence
  Primer
tggccataagtaataccagtatcatatcct

SEQ ID NO: 21 - DNA, 26 bp, Artificial Sequence
  Primer
tgtatggataggtggagaagtcagtg

SEQ ID NO: 22 - DNA, 27 bp, Artificial Sequence
  Primer
tgtgcttatgtggattaccagtaccta

SEQ ID NO: 23 - DNA, 30 bp, Artificial Sequence
  Primer
ttgattctccctcaaaattctcatccaaag

SEQ ID NO: 24 - DNA, 31 bp, Artificial Sequence
  Primer
ttggcaagaaataactcagtagatgatttgc

SEQ ID NO: 25 - DNA, 25 bp, Artificial Sequence
  Primer
ttgttctgcaaagctatcttcccac